(19) United States
(12) Patent Application Publication
(10) Pub. No.: US 2019/0282516 A1
(43) Pub. Date: Sep. 19, 2019

(54) CAROTENOID-CONTAINING COMPOSITION

(71) Applicant: JXTG NIPPON OIL & ENERGY CORPORATION, Tokyo (JP)

(72) Inventor: Masahiro HAYASHI, Tokyo-to (JP)

(73) Assignee: JXTG NIPPON OIL & ENERGY CORPORATION, Tokyo (JP)

(21) Appl. No.: 16/394,289

(22) Filed: Apr. 25, 2019

(30) Foreign Application Priority Data
Apr. 1, 2019 (JP) 2019-070183

(51) Int. Cl. A61K 31/122; A23L 33/12; A23L 5/44; A61K 35/74; A23L 33/135

(52) U.S. Cl. CPC A61K 31/122; A23L 33/12; A23L 5/44; A61K 35/74; A23V 2250/206; A23V 2002/00; A23V 2200/30; A23V 2200/044; A23L 33/135

(57) ABSTRACT

To provide a carotenoid-containing composition obtained by dissolving or dispersing a solid carotenoid derived from a genus Paracoccus microorganism is used.

### CROSS-REFERENCE TO RELATED APPLICATIONS

This application is based upon and claims the benefit of priority from Japanese Patent Application No. 2019-070183, filed on Apr. 1, 2019; the entire contents of which are incorporated herein by reference.

### TECHNICAL FIELD

The present invention relates to a carotenoid-containing composition.

### BACKGROUND ART

A carotenoid is a natural pigment widely existing in the natural world, and astaxanthin, adonirubin, adonixanthin, zeaxanthin, and β-cryptoxanthin, etc., have been known as one of the carotenoids. Astaxanthin, adonirubin, and adonixanthin, etc., have been known to have various effects including an antianxiety physiological action (Patent Literature 1), and are expected to be used as foods, pharmaceuticals, and the like.

Astaxanthin, adonirubin, and adonixanthin, etc., are widely distributed in animals, plant, and microorganisms. For example, astaxanthin is widely distributed in the natural world including fishes such as salmons, trouts, and red seabreams and crustaceans such as crabs, shrimps, and krills, and is also produced by microorganisms such as bacteria belonging to the genus Agrobacterium, the genus Brevibacterium, the genus Paracoccus, the genus Brevundimonas, and the genus Erythrobacter, green algae of the genus Haematococcus, and yeast of the genus Phaffia. Although carotenoids such as astaxanthin and adonixanthin are industrially produced by chemical synthesis methods, carotenoids derived from natural products are required in terms of safety.

Of the above-mentioned microorganisms, bacteria belonging to the genus Paracoccus have advantages such as high productivity of carotenoids, a high growth rate, and easiness of extraction of carotenoids. As an example of astaxanthin-producing strains belonging to the genus Paracoccus, an E-396 strain (FERM BP-4283: dated on Apr. 27, 1993 (date of original deposit), International Patent Organism Depository, National Institute of Advanced Industrial Science and Technology (Central 6, 1-1-1 Higashi, Tsukuba, Ibaraki, Japan)) (Patent Literature 2) has been known. Various methods have been known as a method for obtaining a carotenoid-containing composition purified from the strain. Of these, a method for obtaining a carotenoid-containing composition containing a high content of astaxanthin and a high-purity carotenoid (Patent Literature 3) has been reported.

Here, when carotenoids having various effects are used for various applications, it needs to absorb the carotenoid more efficiently. Therefore, it can be said that a means for improving the bioabsorbability (bioavailability) of carotenoids has been still required.

### CITATION LIST

### Patent Literature

Patent Literature 1: JP 2012-025712 A
Patent Literature 2: JP H08-009964 A
Patent Literature 3: WO 2014/054669 A

### SUMMARY OF THE INVENTION

The present invention provides a novel technical means for effectively improving the bioabsorbability of a carotenoid-containing composition.

This time, the present inventors have found that a carotenoid-containing composition obtained by dissolving or dispersing a solid carotenoid derived from a genus Paracoccus microorganism effectively improves the bioabsorbability of a carotenoid. The present invention is based on such finding.

The present invention includes the following inventions.
(1) A carotenoid-containing composition obtained by dissolving or dispersing a solid carotenoid derived from a genus Paracoccus microorganism.
(2) The composition according to (1), wherein the carotenoid comprises one or more carotenoids selected from astaxanthin, adonirubin, adonixanthin, and a pharmaceutically acceptable salt thereof.
(3) The composition according to (1) or (2), wherein the genus Paracoccus microorganism is Paracoccus carotinifaciens.
(4) The composition according any one of claims 1) to (3), further comprising an orally acceptable or pharmaceutically acceptable additive.
(5) The composition according to any one of (1) to (4) for a human.
(6) The composition according to any one of (1) to (5), which is a food and drink or a food additive or a material thereof.
(7) The composition according to any one of (1) to (6), which is a functional food or a material thereof.
(8) The composition according to any one of (1) to (7), which is a pharmaceutical or a material thereof.
(9) A method for improving an absorbability of a carotenoid, which comprises dissolving or dispersing a solid carotenoid derived from a genus Paracoccus microorganism in the presence of an orally acceptable or pharmaceutically acceptable excipient.

According to the present invention, it is possible to effectively improve the bioabsorbability of a carotenoid by using a carotenoid-containing composition obtained by dissolving or dispersing a solid carotenoid derived from a genus Paracoccus microorganism. The composition of the present invention is advantageous for effectively improving the fast-acting property, persistence, and/or water solubility or water dispersibility of a carotenoid. The composition of the present invention is also advantageous for effectively improving the workability, availability, and/or stability of a carotenoid. The composition of the present invention is also advantageous for reducing the variability in the bioabsorbability of a carotenoid between subjects.

### DETAILED DESCRIPTION OF THE INVENTION

One of the characteristics of a carotenoid-containing composition of the present invention is that it is obtained by dissolving or dispersing a solid carotenoid derived from a genus Paracoccus microorganism.

### Solid Carotenoid

A carotenoid in the present invention is not particularly limited as long as the effect of the present invention is not prevented, and is one or more selected from astaxanthin, adonirubin, adonixanthin, zeaxanthin, β-cryptoxanthin, β-carotene, echinenone, canthaxanthin, 3-hydroxyechinenone, asteroidenone, lycopene, phytoene, phytofluene, neurosporene, and a pharmaceutically acceptable salt thereof, and is preferably astaxanthin, adonirubin, and adonixanthin. Such carotenoid, for example, may be a combination of astaxanthin, adonirubin, and adonixanthin. The carotenoid may be a free form or a fatty acid ester form, and is preferably a free form. The carotenoid may be a stereoisomer such as an optical isomer and a cis-trans isomer. Furthermore, it is preferable to use these carotenoids as an active ingredient.

Astaxanthin is a red pigment and belongs to xanthophyll, which is one of carotenoids. The chemical formula thereof is 3,3′-dihydroxy-β,β-carotene-4,4′-dione (C₄₀H₅₂O₄, molecular weight 596.852), and the structural formula is represented by the following formula:

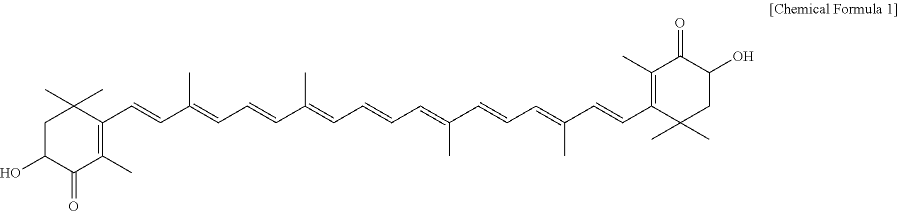 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

Examples of an optical isomer of astaxanthin can include at least one selected from the group consisting of a 3S,3′S-isomer, a 3S,3′R-isomer (meso-isomer), and a 3R,3′R-isomer, and the optical isomer is preferably a 3S,3′S-isomer. Astaxanthin may be a cis isomer or a trans isomer of a conjugated double bond in the center of the molecule or a combination thereof. Examples of the cis isomer include a 9-cis isomer, a 13-cis isomer, a 15-cis isomer, a dicis isomer, or a combination thereof. Astaxanthin is preferably a combination of a cis isomer and a trans isomer.

The chemical formula of adonirubin is 3-hydroxy-β,β-carotene-4,4′-dione (C₄₀H₅₂O₃, molecular weight 580.853), and the structural formula is represented by the following formula:

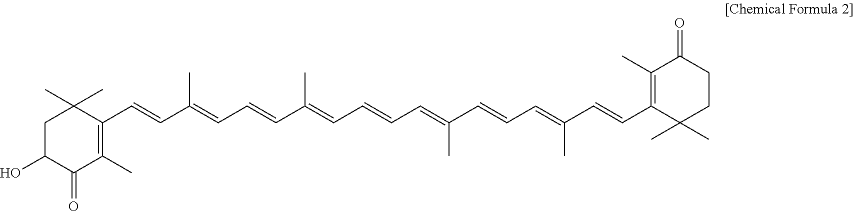 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CCC1=O]

A cis-trans isomer of adonirubin may be a cis isomer, a trans isomer, or a combination thereof. Examples of a cis isomer can include a 13-cis isomer.

The chemical formula of adonixanthin is 3,3′-dihydroxy-β,β-carotene-4-one (C₄₀H₅₄O₃, molecular weight 582.869), and the structural formula is represented by the following formula:

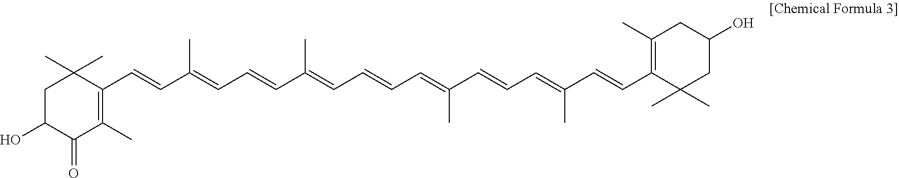 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1]

Examples of an optical isomer of adonixanthin can include at least one selected from the group consisting of a 3S,3′R-isomer, a 3S,3′S-isomer, a 3R,3′S-isomer, and a 3R,3′R-isomer, and the optical isomer is preferably a 3S,3′R-isomer. A cis-trans isomer of adonixanthin may be a cis isomer, a trans isomer, or a combination thereof. The cis-trans isomer of adonixanthin is preferably a combination of a cis isomer and a trans isomer.

In the present invention, the carotenoid may be in a form of a pharmaceutically acceptable salt, and these salts are included in the carotenoid in the present invention. In the present invention, the carotenoid may form a salt with an acid or a base. In the present invention, the pharmaceutically acceptable salt is not particularly limited as long as it forms a pharmaceutically acceptable salt with astaxanthin, adonirubin, adonixanthin, zeaxanthin, β-cryptoxanthin, β-carotene, echinenone, canthaxanthin, 3-hydroxyechinenone, asteroidenone, lycopene, phytoene, phytofluene, and/or neurosporene. Specific example thereof include, but are not limited to, hydrohalides (e.g., hydrofluorides, hydrochlorides, hydrobromates, hydroiodides, etc.), inorganic acid salts (e.g., sulfates, nitrates, perchlorates, phosphates, carbonates, bicarbonates, etc.), organic carboxylates (e.g., acetates, oxalates, maleates, tartrates, fumarates, citrates, etc.), organic sulfonates (e.g., methanesulfonates, trifluoromethanesulfonates, ethanesulfonates, benzenesulfonates, toluenesulfonates, camphorsulfonates, etc.), amino acid salts (e.g., aspartates, glutamates, etc.), quaternary amine salts, alkali metal salts (e.g., sodium salts, potassium salts, etc.), and alkaline earth metal salts (e.g., magnesium salts, calcium salts, etc.), etc.

A solid carotenoid of the present invention is not particularly limited as long as it is a solid of a carotenoid that can be normally used for pharmaceuticals, foods, and the like. Examples of such solid carotenoid include a crystal of a carotenoid and a mixed solid with a carotenoid and other components, and the solid carotenoid is preferably a dried crystal of a carotenoid. The purity of the carotenoid in the solid carotenoid is preferably high, and, for example, is 30% by mass or more, preferably 30 to 100% by mass, more preferably 50 to 100% by mass, and still more preferably 80 to 100% by mass.

The content of the carotenoid in the composition of the present invention is not preferably limited as long as the effect of the present invention is not prevented, and, for example, is 0.01 to 99% by mass, preferably 0.5 to 70% by mass, more preferably 0.7 to 50% by mass, and still more preferably 1 to 40% by mass, based on the whole composition. The content of astaxanthin, adonirubin, adonixanthin, zeaxanthin, β-cryptoxanthin, β-carotene, echinenone, canthaxanthin, 3-hydroxyechinenone, asteroidenone, lycopene, phytoene, phytofluene, and neurosporene in the composition of the present invention is measured by high performance liquid chromatography (HPLC method). Such measurement can be simply performed by using a commercial device and column (e.g., Wakosil-II SIL-100 (φ4.6×250 mm) (manufactured by Wako Pure Chemical Industries, Ltd.)). The above-mentioned measurement can be performed by, for example, flowing an n-hexane/tetrahydrofuran/methanol mixture (volume ratio of 40:20:1), which is a mobile phase, at a flow rate of 1.0 mL/min at a constant temperature near room temperature. Detection of astaxanthin, adonirubin, adonixanthin, zeaxanthin, β-cryptoxanthin, β-carotene, echinenone, canthaxanthin, 3-hydroxyechinenone, asteroidenone, lycopene, and neurosporene are performed at a wavelength of 470 nm. Detection of phytoene and phytofluene is performed at a wavelength of 288 nm.

As the solid carotenoid of the present invention, a microorganism-derived substance (naturally-derived substance) produced by fermentation methods with microorganisms or extraction and purification methods from microorganisms, or the like can be used. The microorganism-derived substance as used herein is a product obtained from microorganisms, and preferably a genus Paracoccus microorganism-derived substance. Here, as the genus Paracoccus microorganisms, Paracoccus carotinifaciens, Paracoccus marcusii, Paracoccus haeundaensis, and Paracoccus zeaxanthinifaciens are preferably used, and more preferably Paracoccus carotinifaciens is used. Specific examples of the strain of the genus Paracoccus microorganisms include Paracoccus carotinifaciens E-396 strain and genus Paracoccus bacteria A581-1 strain (FERM BP-4671), and these mutants are also preferably used in the present invention.

Examples of a method for culturing genus Paracoccus microorganisms (i.e., a method for producing a carotenoid from genus Paracoccus microorganisms), and a method for extracting and purifying a solid carotenoid include the following methods.

The method for culturing genus Paracoccus microorganisms is not particularly limited as long as it is a method that can culture such microorganisms. For example, the culture can be performed in accordance with the method mentioned in Example 1 of JP 2007-319015 A. The method will be briefly mentioned below.

A medium containing glucose, a meat extract, peptone, sodium chloride, etc., is put into a test tube, and is steam-pasteurized. To this, an E-396 strain (FERM BP-4283) is inoculated, and reciprocal shaking culture is performed. This culture medium is centrifuged and then freeze-dried to obtain dried bacterial cells containing a carotenoid.

The method for extracting and purifying a solid carotenoid is not particularly limited as long as it is a method that can extract and purify a solid carotenoid. For example, the extraction and purification can be performed in accordance with the method mentioned in WO2014/054669. The method will be briefly mentioned below.
<Ethanol extraction step> Ethanol is added to the dried bacterial cells of a genus Paracoccus microorganism containing a carotenoid obtained above, and the carotenoid is extracted while stirring in a high-pressure container under nitrogen atmosphere at about 90° C. After the solution temperature is cooled (e.g., 65° C.), the pressure container is opened, the bacterial cells are removed from the extract by filtration, and further the bacterial cell cake is washed with ethanol to obtain an extract containing a carotenoid.
<Step of concentrating and crystallizing the extract> Regarding the extract obtained by the ethanol extraction step mentioned above, part of ethanol is distilled away using a rotary evaporator with adjusted reduced pressure, and the solid content of the extract is concentrated (e.g., 1.25 to 20 times). Then, this concentrated solution is matured overnight (e.g., 30° C.) to precipitate a crystal.
<Step of collecting by filtration and drying the crystal> From the solution containing the crystal obtained in the step of concentrating and crystallizing the extract mentioned above, the crystal is recovered by filtration. The recovered crystal is dried under reduced pressure (e.g., 2 hours at 100° C.) to obtain a dried crystal (solid carotenoid).

### Orally Acceptable or Pharmaceutically Acceptable Additive

The carotenoid-containing composition of the present invention is not limited as long as a solid carotenoid derived from a genus Paracoccus microorganism is dissolved or dispersed, and preferably further contains an orally acceptable or pharmaceutically acceptable additive. Examples of the additive mentioned above include solvents, dispersion media, solubilizing agents, solubilizers, lubricants, emulsifiers, isotonizing agents, stabilizers, preservatives, antioxidants (antioxidants), antiseptics, surfactants, adjusters, chelating agents, pH adjusters, buffers, excipients, thickeners, thickening stabilizers, enzymes, seasonings, reinforcing agents, manufacturing agents, raising agents, disintegrators, coating agents, gum bases, glazing agents, sweeteners, acidulants, bittering agents, coloring agents, extraction solvents, coagulants for tofu, fungicides, humectants, bleaching agents, aromatics, perfumes, anti-caking agents, antifoaming agents, bulking agents, carbonating agents, processing aids, emulsifying salts, firming agents, flavor enhancers, flour treatment agents, foaming agents, gelling agents, fillers, antiseptics, propellants, sequestrants, flavors, food contact substances, drying agents, color couplers, nutrient supplements, oxidizing agents, reducing agents, solvents, lubricants, mold separating agents, synergists, texturizers, fumigants, cleaning agents, cooling/freezing agents. Specific additives will be mentioned below, and since the above-mentioned classification is based on each application, the same substance is sometimes classified as an additive for another application.

Examples of the above-mentioned solvent used in the present invention include a lipophilic liquid, and such liquid is preferably a liquid at room temperature. The lipophilic liquid may be one lipophilic substance or a combination of two or more lipophilic substances.

Examples of the lipophilic substance contained in the lipophilic liquid include an oily solvent, a silicone oil, and an oil-soluble biologically active substance. In other words, each of the lipophilic liquid, the oily solvent, and the oil-soluble biologically active substance may be oil.

Examples of the oily solvent include vegetable oils and fats such as rapeseed oil, palm oil, soybean oil (e.g., partially hydrogenated soybean oil), olive oil, jojoba oil, coconut oil, safflower oil, corn oil, sunflower oil, peanut oil, palm kernel oil, cottonseed oil, cocos oil, wheat oil, orange oil, peppermint oil, oleoresin, citrus oil, elemi resin, and mastic resin; animal oils and fats such as beef tallow and lard; and other oily solvents such as sucrose acetate isobutyrate (SAIB), rosin, dammar resin, ester gum, glycerol fatty acid ester, and medium-chain triglyceride (MCT); and a combination thereof. Preferable oily solvents comprise medium-chain triglyceride, sucrose acetate isobutyrate, and vegetable oils and fats. Here, the number of carbon atoms of medium-chain triglyceride (MCT) is not limited. MCT refers to, for example, triacylglycerol composed of medium chain fatty acid with about 6 to 12 carbon atoms, preferably 6 to 10 carbon atoms, and more preferably 8 to 10 carbon atoms, and generally commercially available one can be used without limitation. Specific examples thereof include caprylic acid triglyceride, capric acid triglyceride, caprylic acid and capric acid mixed triglyceride, etc., and a mixture thereof.

Examples of the oil-soluble biologically active substance include fat-soluble vitamins such as liver oil, vitamin A (e.g., retinol, etc.), vitamin A oil, vitamin D (e.g., ergocalciferol, cholecalciferol, etc.), vitamin B2-butyrate, fatty acid ester of ascorbic acid, vitamin E (e.g., tocopherol, tocotrienol, etc.), vitamin K (e.g., phylloquinone, menaquinone, etc.), and a derivative thereof, for example, vitamin A ester and vitamin E ester (retinyl acetate, tocopherol acetate, etc.); vegetable essential oils such as limonene, linalool, nerol, citronellol, geraniol, citral, l-menthol, eugenol, cinnamic aldehyde, anethole, perillaldehyde, vanillin, and γ-undecalactone; resveratrol, oil-soluble polyphenol, glycosylceramide, sesamin, phosphatidylserine, coenzyme Q 10, ubiquinol, α-lipoic acid; ω-3 fatty acids such as arachidonic acid, α-linolenic acid, eicosapentaenoic acid, and docosahexaenoic acid; ω-6 fatty acids such as linoleic acid and γ-linolenic acid; and plant sterol. The oil-soluble biologically active substance may be one alone or a combination of two or more oil-soluble biologically active substances.

The above-mentioned solvent used in the present invention may be further alcohol, ether, ester, ketone, acetal, halogenated carbon, alkane, nitrous oxide, oil and fat, carbon dioxide, water, and a combination thereof. Specific examples thereof include methanol, ethanol, n-propanol, isopropanol, 1,2-butanediol 1-methyl ether(1-methoxybutane-2-ol), 1,2-propanediol 1-n-propyl ether(1-propoxypropane-2-ol), tetrahydrofuran, methyl ethyl ketone, glycerin, ethyl acetate, methyl acetate, diethyl ether, cyclohexane, food oil and fat, nitrous oxide, 1,1,1,2-tetrafluoroethane, 1,1,2-trichloroethene, carbon dioxide, 1-butanol, 2-butanol, butane, propane, propylene glycol, water, acetone, dichloromethane, chloroform, and a combination thereof.

Examples of the above-mentioned dispersion medium used in the present invention include ω-3 fatty acids such as arachidonic acid, α-linolenic acid, eicosapentaenoic acid, and docosahexaenoic acid; vegetable oils and fats; gases such as nitrogen, air, and carbon dioxide; and aqueous solvents, and the dispersion medium is preferably water, eicosapentaenoic acid, and docosahexaenoic acid. When gas is used as the dispersion medium, high-pressure gas (e.g., high-pressure nitrogen, air, and carbon dioxide) is preferred. By dispersing a solid or liquid solid carotenoid composition (preferably, a microparticle or a minute droplet) to such gas, it is possible to make a spray product such as an aerosol agent.

Examples of the above-mentioned pH adjuster used in the present invention include carbon dioxide.

Examples of the above-mentioned disintegrator used in the present invention include carbon dioxide, nitrogen, and helium. According to one embodiment of the present invention, it is preferable that carbon dioxide, nitrogen, helium, etc., are filled in the cavity or space in a solid composition such as a granule and a beadlet (microcapsule), or it is preferable to make a cavity or space by carbon dioxide, nitrogen, helium, etc., when the solid composition is produced. Such composition is advantageous in terms of the fact that it easily disintegrates when water is added or taken.

Examples of the above-mentioned thickener used in the present invention include pectin, carrageenan, modified starch, modified cellulose, xanthan gum, acacia gum, pectin, guar, carob gum, gellan gum, carboxymethyl cellulose, locust gum, agar, propylene glycol, psyllium seed gum, curdlan, tamarind gum, tara gum, polyacrylate, and alginate, and the thickener is preferably sugar beet pectin.

Examples of the above-mentioned excipient used in the present invention include sugars. Suitable examples of the above-mentioned sugar include monosaccharides, disaccharides, oligosaccharides, or polysaccharides. Examples of the monosaccharide or disaccharide include sucrose, invert sugars, glucose, fructose, lactose, xylose, arabinose, ribose and maltose, and trehalose. The monosaccharide or disaccharide may be as it is or may be in the form of syrup, for example, fructose syrup or glucose syrup. Examples of the oligosaccharide or polysaccharide include starch and starch derivatives such as starch hydrolysates, for example, dextrin and maltodextrin particularly in the range of 5 to 65 dextrose equivalent (hereinafter referred to as DE), and glucose syrup particularly in the range of 20 to 95 DE. One sugar may be used, or a mixture of two or more sugars may be used.

Examples of the antioxidant used in the present invention include water-soluble antioxidants and fat-soluble antioxidants. Examples of the water-soluble antioxidant include ascorbic acid and a salt thereof (e.g., sodium ascorbate, etc.), citric acid, and sodium citrate. Examples of the fat-soluble antioxidant include d-α-tocopherol (i.e., natural tocopherol), β- and γ-tocopherol, and a mixture thereof; ascorbic acid esters of fatty acid such as ascorbyl palmitate or stearate; butylhydroxytoluene; butylated hydroxyanisole; propyl gallate; or t-butylhydroxyquinoline;6-ethoxy-1,2-dihydroxy-2,2,4-trimethylquinoline (ethoxyquin), 3,5-di-t-4-butylhydroxytoluene (BHT), and 3-t-butylated hydroxyanisole (BHA). As the antioxidant, carbon dioxide or nitrogen may be used. One antioxidant may be used, or a mixture of two or more antioxidants may be used.

Examples of the above-mentioned emulsifier used in the present invention include gelatin, modified starch, ascorbyl palmitate, polyglycerol ester of fatty acid, propylene glycol ester of fatty acid, phospholipid, whey protein, whey protein hydrolysate, sucrose fatty acid ester, and a combination thereof. Gelatin is an emulsifier that simultaneously acts as a texture agent. Optional gelatin that exhibits a “bloom” substantially in the range of 0 to about 300 can be used. Modified starch is a processed product of starch that was produced chemically or enzymatically, and examples thereof include starch ether, starch ester, and phosphoric acid starch. Examples of the starch ester include starch octenyl succinate, and the starch ester is preferably starch sodium octenyl succinate. Examples of the starch sodium octenyl succinate include Capsul (registered trademark) (starch sodium octenyl succinate) and Purity (registered trademark) Gum 2000 (starch sodium octenyl succinate) manufactured by National Starch. Examples of the polyglycerol ester of fatty acid include polyglycerol-3 polyricinoleate (PGPR 90), sorbitan ester of fatty acid, for example, sorbitan monostearate (span 60), and PEG (20) sorbitan monooleate. Examples of the phospholipid include lecithin. As the sucrose fatty acid ester, sucrose fatty acid ester with an HLB value of 10 to 18 is preferred, and such sucrose fatty acid ester includes sucrose stearate, sucrose palmitate, sucrose myristate, sucrose laurate, and sucrose oleate.

Examples of the above-mentioned humectant used in the present invention include glycerin, sorbitol, polyethylene glycol, propylene glycol, and a combination thereof.

Examples of the above-mentioned manufacturing agent used in the present invention include calcium silicate, cyclodextrin, a water-soluble cyclodextrin derivative, and a combination thereof. Examples of the cyclodextrin include α-cyclodextrin, β-cyclodextrin, γ-cyclodextrin, and a combination thereof. Examples of the water-soluble cyclodextrin derivative include hydroxypropyl-β-cyclodextrin, methyl-β-cyclodextrin, nnonoacetyl-β-cyclodextrin, triacetyl-β-cyclodextrin, nnonochlorotriazyl-β-cyclodextrin, sulfobutyl ether-β-cyclodextrin, di-O-methyl-β-cyclodextrin, maltosyl-β-cyclodextrin, and a combination thereof.

Examples of the stabilizer used in the present invention include methyl 4-hydroxybenzoate, propyl 4-hydroxybenzoate, sorbic acid or benzoic acid, or a salt thereof and a combination thereof in order to increase the stability of the composition for microbial degradation.

The additives used in the present invention include food additives such as existing additives and designated additives, in addition to the above. Examples of the specific existing additives below (in parentheses indicate the use): aureobasidium cultured (thickening stabilizers), agarase (enzymes), actinidine (enzymes), agrobacterium succinoglycan (thickening stabilizers), acylase (enzymes), ascorbate oxidase (enzymes), L-asparagine (seasonings, reinforcing agents), L-aspartic acid (seasonings, reinforcing agents), aspergillus terreus glycoprotein (manufacturing agents), α-acetolactate decarboxylase (enzymes), 5′-adenylic acid (reinforcing agents), annatto extract (coloring agents), linseed gum (thickening stabilizers), aminopeptidase (enzymes), α-amylase (enzymes), β-amylase (enzymes), L-alanine (seasonings, reinforcing agents), gum arabic (thickening stabilizers), arabino galactan (thickening stabilizers), L-Arabinose (sweeteners), L-arginine (seasonings, reinforcing agents), alginic acid (thickening stabilizers), alginate lyase (enzymes), aluminium (coloring agents), anthocyanase (enzymes), isoamylase (enzymes), iso-α-bitter acid (bittering agents), isomaltodextranase (enzymes), itaconic acid (acidulants), rice straw ash extract (manufacturing agents), inulinase (enzymes), inositol (reinforcing agents), invertase (enzymes), welan gum (thickening stabilizers), turmeric oleoresin curcumin (coloring agents), urushi wax (gum bases, glazing agents), urease (enzymes), exonnaltotetraohydrolase (enzymes), esterase (enzymes), elemi resin (thickening stabilizers, gum bases), sodium chloride-decreased brine(saline lake) (seasonings), ozokerite (gum bases), ozone (manufacturing agents), oligogalacturonic acid (manufacturing agents), γ-oryzanol (antioxidants), oregano extract (manufacturing agents), orange colour (coloring agents), seaweed ash extract (manufacturing agents), kaolin (manufacturing agents), cacao colour (coloring agents), japanese persimmon colour (coloring agents), granite porphyry (manufacturing agents), cassia gum (thickening stabilizers), catalase (enzymes), active carbon (manufacturing agents), activated acid clay (manufacturing agents), gum ghatti (thickening stabilizers), catechin (antioxidants), curdlan (thickening stabilizers, manufacturing agents), caffeine(extract) (bittering agents, etc.), carrageenan(semirefined carrageenan, purified carrageenan, powdered red algae) (thickening stabilizers), α-galactosidase (enzymes), β-galactosidase (enzymes), mustard extract (manufacturing agents), caramel I (coloring agents, manufacturing agents), caramel II (coloring agents, manufacturing agents), caramel III (coloring agents manufacturing agents), caramel IV (coloring agents, manufacturing agents), karaya gum (thickening stabilizers), carnauba wax (gum bases, glazing agents), carboxypeptidase (enzymes), carob germ colour (coloring agents, manufacturing agents), carob bean gum (thickening stabilizers), rumput roman extract (preservatives), licorice extract (sweeteners), licorice oil extract (antioxidants), candelilla wax (gum bases, glazing agents), xanthan gum (thickening stabilizers), xylanase (enzymes), D-xylose (sweeteners), chitinase (enzymes), chitin (thickening stabilizers), chitosanase (enzymes), chitosan (thickening stabilizers, manufacturing agents), red bark cinchona extract (bittering agents, etc.), phellodendron bark extract (bittering agents, etc.), fish scale foil (coloring agents), quillaia extract (emulsifiers), gold (coloring agents, manufacturing agents), silver (coloring agents), guar gum (thickening stabilizers), enzymatically hydrolyzed guar gum (thickening stabilizers), guaiac resin (antioxidants), guajac resin (gum bases), quercetin (antioxidants), gardenia blue (coloring agents), gardenia red (coloring agents), gardenia yellow (coloring agents), gutta hang kang (gum bases), gutta percha (gum bases), cristobalite (manufacturing agents), glucanase (enzymes), glucoamylase (enzymes), glucosamine (thickening stabilizers, manufacturing agents), α-glucosidase (enzymes), β-glucosidase (enzymes), α-glucosyltransferase (enzymes), α-glucosyltransferase treated stevia (sweeteners), glucose isomerase (enzymes), glucose oxidase (enzymes), glutaminase (enzymes), L-glutamine (seasonings, reinforcing agents), grapefruit seed extract (manufacturing agents), kooroo colour (coloring agents), clove extract (antioxidants), chlorophylline (coloring agents), chlorophyll (coloring agents), smoke flavourings (wood vinegar, liquid smoke) (manufacturing agents), diatomaceous earth (manufacturing agents), gentian root extract (bittering agents, etc.), higher fatty acid (manufacturing agents), spice extract (bittering agent , etc.), enzymatically modified isoquercitrin (antioxidants), enzymatically modified naringin (bittering agents, etc.), enzymatically modified hesperidin (reinforcing agents), enzymatically modified rutin (extract) (antioxidants, reinforcing agents, coloring agents), enzymatically modified lecithin (emulsifiers), enzymatically hydrolyzed licorice extract (sweeteners), enzymatically decomposed apple extract (antioxidants), enzymatically decomposed lecithin (emulsifiers), yeast cell wall (thickening stabilizers, manufacturing agents), kaoliang color (coloring agents), cochineal extract (coloring agents), bone charcoal (manufacturing agents), bone carbon black (manufacturing agents), sesame seed oil unsaponified matter (antioxidants), sesame straw ash extract (manufacturing agents), rubber (gum bases), resin of depolymerized natural rubber (gum bases), rice bran oil extract (antioxidants), enzymatically decomposed rice bran (antioxidants), rice bran wax (gum bases, glazing agents), psyllium seed gum (thickening stabilizers), cane wax (gum bases), artemisia sphaerocephala seed gum artemisia seed gum (manufacturing agents, thickening stabilizers), acid clay (manufacturing agents), acid phosphatase (enzymes), oxygen (manufacturing agents), shea nut colour (coloring agents), cyanocobalamin (reinforcing agents), shellac (gum bases, glazing agents), shellac wax (gum bases, glazing agents), gellan gum (thickening stabilizers), jelutong (gum bases), cyclodextrin (manufacturing agents), cyclodextrin glucanotransferase (enzymes), L-cystine (seasonings, reinforcing agents), perilla extract (manufacturing agents), sandalwood red (coloring agents), 5′-cytidylic acid (reinforcing agents), jamaica quassia extract (bittering agents, etc.), ginger extract (manufacturing agents), calcinated calcium(calcinated sea urchin shell calcium, calcinated shell calcium, calcinated bone calcium, calcinated coral calcium, tricalcium phosphate, calcinated eggshell calcium) (reinforcing agents, manufacturing agents), vegetable sterol (emulsifiers), vegetable carbon black (coloring agents), vegetable lecithin (emulsifiers), milt protein (preservatives), hydrogen (manufacturing agents), stevia extract (sweeteners), powdered stevia (sweeteners), spirulina color (coloring agents), sphingolipid (emulsifiers), quicklime (manufacturing agents), essential oil-removed fennel extract (antioxidants), horseradish extract (antioxidants, manufacturing agents), zein (manufacturing agents), zeolite (manufacturing agents), sage extract (antioxidants), sepiolite (manufacturing agents), L-serine (seasonings, reinforcing agents), cellulase (enzymes), crude potassium chloride (sea water) (seasonings), crude magnesium chloride (sea water) (manufacturing agents), buckwheat ash extract (manufacturing agents), sorva (gum bases), sorvinha (gum bases), soybean saponin (emulsifiers), thaumatin (sweeteners), taurine (extract) (seasonings), onion color (coloring agents), tamarind color (coloring agents), tamarind seed gum (thickening stabilizers), tara gum (thickening stabilizers), talc (gum bases, manufacturing agents), powdered bile (emulsifiers), amino acid-sugar reaction product (antioxidants), tannase (enzymes), tannin (extract) (tannin of persimmon, vegetable tannin, tannin of silver wattle) (manufacturing agents), chicle (gum bases), nitrogen (manufacturing agents), tea dry distillate (manufacturing agents), tea extract (antioxidants, manufacturing agents), chilte (gum bases), L-tyrosine (seasonings, reinforcing agents), tunu (gum bases), thujaplicin (extract) (preservatives), 5′-deaminase (enzymes), depolymerized natural rubber (gum bases), theobromine (bittering agents, etc.), dextranase (enzymes), dextran (thickening stabilizers), iron (reinforcing agents, manufacturing agents), dunaliella carotene (reinforcing agents, coloring agents), copper (manufacturing agents), paprika color (coloring agents), capsicum water-soluble extract (manufacturing agents), cholesterol (emulsifiers), tocotrienol (antioxidants), d-α-tocopherol (antioxidants, reinforcing agents), d-β-tocopherol (antioxidants, reinforcing agents), d-δ-tocopherol (antioxidants, reinforcing agents), tomato color (coloring agents), tragacanth gum (thickening stabilizers), transglucosidase (enzymes), transglutaminase (enzymes), trypsin (enzymes), trehalose (manufacturing agents), trehalose phosphorylase (enzymes), tororoaoi (thickening stabilizers), bacillus natto gum (thickening stabilizers), petroleum naphtha (manufacturing agents), coffee bean extract (antioxidants), naringinase (enzymes), naringin (bittering agents, etc.), niger gutta (gum bases), absinth extract (bittering agents, etc.), nickel (manufacturing agents), carrot carotene (reinforcing agents, coloring agents), roasted rice bran extract (manufacturing agents), roasted soybean extract (manufacturing agents), peroxidase (enzymes), platinum (manufacturing agents), papain (enzymes), palm oil carotene (reinforcing agents, coloring agents), perlite (manufacturing agents), Palladium (manufacturing agents), paraffin wax (gum bases, glazing agents), pancreatin (enzymes), hyaluronic acid (manufacturing agents), microcrystalline cellulose (manufacturing agents), microfibrillated cellulose (thickening stabilizers, manufacturing agents), L-histidine (seasonings, reinforcing agents), beet red (coloring agents), L-hydroxyproline (seasonings, reinforcing agents), sunflower seed extract (antioxidants), vermiculite (manufacturing agents), furcellaran (thickening stabilizers), phaffia colour (coloring agents), ficin (enzymes), phytase (enzymes), phytic acid (acidulants, manufacturing agents), phytin (extract) (manufacturing agents), ferritin (reinforcing agents), ferulic acid (antioxidants), fukuronori extract (thickening stabilizers), butane (manufacturing agents), grape skin color (coloring agents), grape skin-derived substance (manufacturing agents), grape seed extract (antioxidants, manufacturing agents), brazilian licorice extract (sweeteners), fructosyl transferase (enzymes), pullulanase (enzymes), pullulan (thickening stabilizers, manufacturing agents), protease (enzymes), propane (manufacturing agents), propolis extract (antioxidants), bromelain (enzymes), L-proline (seasonings, reinforcing agents), fractionated lecithin (antioxidants), powdered cellulose (manufacturing agents), powdered rice hulls (gum bases), Pecan nut colour (coloring agents), hexane (manufacturing agents), pectinase (enzymes), pectin (thickening stabilizers), pectin digests (preservatives), hego-ginkgo leaf extract (antioxidants), hesperidinase (enzymes), hesperidin (reinforcing agents), betaine (seasonings), monascus yellow (coloring agents), monascus color (coloring agents), carthamus red (coloring agents), carthamus yellow (coloring agents), venezuelan chicle (gum bases), pepsin (enzymes), heptane (manufacturing agents), Peptidase (enzymes), haematococcus algae color (coloring agents), hemicellulose (enzymes), heme iron (reinforcing agents), helium (manufacturing agents), bentonite (manufacturing agents), phosphodiesterase (enzymes), phospholipase (enzymes), gallic acid (antioxidants), Jojoba wax (gum bases), polyphenol oxidase (enzymes), ε-polylysine (preservatives), microcrystalline wax (gum bases, glazing agents), macrophomopsis gum (thickening stabilizers), mastic gum (gum bases), massaranduba chocolate (gum bases), massaranduba balata (gum bases), marigold color (coloring agents), maltose phosphorylase (enzymes), maltotriohydrolase (enzymes), non-calcinated calcium (non-calcinated shellcalcium, non-calcinated bone calcium, non-calcinated coral calcium, non-calcinated mother-of-pearl layer calcium, non-calcinated eggshell calcium) (reinforcing agents), mixed tocopherols (antioxidants, reinforcing agents), bees wax (gum bases, glazing agents), myrrh (gum bases), purple sweet potato color (coloring agents), purple corn color (coloring agents), purple yam colour (coloring agents), muramidase, (enzymes), menaquinone (extract) (reinforcing agents), mevalonic acid (manufacturing agents), melaleuca oil (antioxidants), mousouchiku dry distillate (manufacturing agents), mousouchiku extract (manufacturing agents), wood chip (manufacturing agents), charcoal (manufacturing agents), japan wax (gum bases, glazing agents), timber ash (manufacturing agents), timber ash extract (manufacturing agents), peach gum (thickening stabilizers), chinese bayberry extract (antioxidants), yucca foam extract (emulsifiers, manufacturing agents), luohanguo extract (sweeteners), lactoperoxidase (enzymes), lactoferrin concentrates (manufacturing agents), lac colour (coloring agents), lanolin (gum bases, glazing agents), rhamsan gum (thickening stabilizers), L-rhamnose (sweeteners), yolk lecithin (emulsifiers), L-lysine (seasonings, reinforcing agents), lysozyme (enzymes), lipase (enzymes), lipoxygenase (enzymes), D-ribose (sweeteners), liquid paraffin (manufacturing agents), linter cellulose (manufacturing agents), enzymatically decomposed rutin (antioxidants), rutin(extract)(azuki extract, japanese pagoda tree extract, buckwheat extract) (antioxidants, coloring agents), ruthenium (manufacturing agents), mannentake extract (bittering agents, etc.), leche de vaca (gum bases), levan (thickening stabilizers), rennet (enzymes), L-leucine (seasonings, reinforcing agents), logwood color (coloring agents), rosidinha (gum bases), rosin (gum bases), rosemary extract (antioxidants).

In addition, examples of the specific designated additives below (in parentheses indicate the use):
zinc salts (zinc gluconate) (zinc sulfate) (reinforcing agents), chlorous acid water (disinfectants), sodium chlorite (bleaching agents, disinfectants), nitrous oxide (manufacturing agents), adipic acid (acidulants), sodium nitrite (color couplers), L-ascorbic acid (reinforcing agents, antioxidants), calcium L-ascorbate (reinforcing agents, antioxidants), L-ascorbic acid 2-glucoside (reinforcing agents), L-ascorbic stearate (reinforcing agents, antioxidants), sodium L-ascorbate (reinforcing agents, antioxidants), L-ascorbic palmitate (reinforcing agents, antioxidants), asparaginase (enzymes), monosodium L-aspartate (seasonings, reinforcing agents), aspartame (sweeteners), acesulfame potassium (acesulfame K) (sweeteners), acetylated distarch adipate (thickening stabilizers, manufacturing agents), acetylated oxidized starch (thickening stabilizers, manufacturing agents), acetylated distarch Phosphate (thickening stabilizers, manufacturing agents), acetaldehyde (perfumes), ethyl acetoacetate (perfumes), acetophenone (perfumes), acetone (manufacturing agents), sodium selenite (reinforcing agents), azoxystrobin (fungicides), advantame (sweeteners), anisaldehyde (perfumes), β-apo-8′-carotenal (coloring agents), (3-Amino-3-carboxypropyl) dimethyl sulfonium chloride (perfumes), amylalcohol (perfumes), α-amyl cinnam aldehyde(a-amyl cinnamic aldehyde) (perfumes), DL-alanine (seasonings, reinforcing agents), sodium sulfite (preservatives, antioxidants, bleaching agents), L-arginine L-glutamate (seasonings, reinforcing agents), ammonium alginate (thickening stabilizers), potassium alginate (thickening stabilizers), calcium alginate (thickening stabilizers), sodium alginate (thickening stabilizers), propylene glycol alginate (thickening stabilizers), benzoic acid (preservatives), sodium benzoate (preservatives), methyl anthranilate (perfumes), ammonia (manufacturing agents), ammonium isovalerate (perfumes), ionone (perfumes), ion exchange resin (manufacturing agents), isoamylalcohol (perfumes), isoeugenol (perfumes), isoamyl isovalerate (perfumes), ethyl isovalerate (perfumes), isoquinoline (perfumes), isothiocyanates (perfumes), allyl isothiocyanate (perfumes), isovaleraldehyde (perfumes), isobutanol (perfumes), isobutylaldehyde (perfumes), isopropanol (perfumes, extraction solvents), isopentylamine (perfumes), L-isoleucine (seasonings, reinforcing agents), disodium 5′-inosinate (seasonings), imazalil (fungicides), indoles and its derivatives (perfumes), disodium 5′-uridylate (sodium 5′-uridylate) (seasonings), γ-undecalactone (perfumes), ester gum (gum bases), esters (perfumes), mixture of 2-ethyl-3,5-dimethylpyrazine and 2-ethyl-3,6-dimethyl pyrazine (perfumes), ethylvanillin (perfumes), 2-ethylpyrazine (perfumes), 3-ethylpyridine (perfumes), 2-ethyl-3-methylpyrazine (perfumes), 2-ethyl-5-methylpyrazine (perfumes), 2-ethyl-6-methylpyrazine (perfumes), 5-ethyl-2-methylpyridine (perfumes), calcium disodium ethylene diamine tetra acetate(calcium disodium EDTA) (antioxidants), disodium ethylenediaminetetraacetate (disodiumEDTA) (antioxidants), ethers (perfumes), erythorbic acid (isoascorbic acid) (antioxidants), sodium erythorbate (antioxidants), ergocalciferol (reinforcing agents), ammonium chloride (manufacturing agents), potassium chloride (seasonings), calcium chloride (reinforcing agents, coagulants for tofu), ferric chloride (reinforcing agents), magnesium chloride (reinforcing agents, manufacturing agents), hydrochloric acid (manufacturing agents), eugenol (perfumes), octanal (perfumes), octanoic acid (disinfectants (component of peracetic acid formulation)), ethyl octanoate (perfumes), starch sodium octenyl succinate (thickening stabilizers, emulsifiers, manufacturing agents), o-phenylphenol and sodium o-phenylphenate (fungicides), sodium oleate (coating agents), peracetic Acid (disinfectants (component of peracetic acid formulation)), hydrogen peroxide (bleaching agents, disinfectants), benzoyl peroxide (flour treatment agents), sodium caseinate (manufacturing agents), ammonium persulfate (flour treatment agents), calcium carboxymethylcellulose (thickening stabilizers), sodium carboxymethylcellulose (thickening stabilizers), β-carotene (coloring agents, reinforcing agents), canthaxanthin (coloring agents), isoamyl formate (perfumes), geranyl formate (perfumes), citronellyl formate (perfumes), xylitol (sweeteners), disodium 5′-guanylate (seasonings), citric acid (acidulants), isoprocpyl citrate (antioxidants), triethyl citrate (perfumes, emulsifiers), monopotassium citrate and tripotassium citrate (seasonings), calcium citrate (seasonings, reinforcing agents, manufacturing agents), sodium ferrous citrate (sodium iron citrate) (reinforcing agents), ferric citrate (reinforcing agents), ferric ammonium citrate (reinforcing agents), trisodium citrate (acidulants, seasonings), glycine (seasonings, reinforcing agents, manufacturing agents), glycerol (glycerin) (manufacturing agents), glycerol esters of fatty acids (emulsifiers, gum bases, manufacturing agents), calcium glycerophosphate (reinforcing agents), disodium glycyrrhizinate (sweeteners), glucono-delta-lactone (acidulants, manufacturing agents), gluconic acid (acidulants), potassium gluconate (acidulants, seasonings, manufacturing agents), calcium gluconate (reinforcing agents), ferrous gluconate (iron gluconate) (reinforcing agents), sodium gluconate (acidulants, seasonings, manufacturing agents), glutamyl-valyl-glycine (seasonings), L-glutamic acid (seasonings, reinforcing agents), monoammonium L-glutamate (seasonings), monopotassium L-glutamate (seasonings, reinforcing agents), monocalcium Di-L-Glutamate (seasonings, reinforcing agents), monosodium L-glutamate (seasonings, reinforcing agents), monomagnesium Di-L-glutamate (seasonings, reinforcing agents), calcium silicate (manufacturing agents), magnesium silicate (manufacturing agents), cinnamic acid (perfumes), ethyl cinnamate (perfumes), methyl cinnamate (perfumes), ketones (perfumes), geraniol (perfumes), high test hypochlorite (manufacturing agents), succinic acid (acidulants, seasonings), monosodium succinate (acidulants, seasonings), disodium succinate (acidulants, seasonings), cholecalciferol (reinforcing agents), sodium chondroitin sulfate (manufacturing agents), isoamyl acetate (perfumes), ethyl acetate (perfumes, manufacturing agents), calcium acetate (reinforcing agents), geranyl acetate (perfumes), cyclohexyl acetate (perfumes), citronellyl acetate (perfumes), cinnamyl acetate (perfumes), terpinyl acetate (perfumes), starch acetate (thickening stabilizers, manufacturing agents), sodium acetate (acidulants, seasonings, manufacturing agents), polyvinyl acetate (gum bases, coating agents), phenethyl acetate (phenylethyl acetate) (perfumes), butyl acetate (perfumes), benzyl acetate (perfumes), l-menthyl acetate (perfumes), linalyl acetate (perfumes), saccharin (sweeteners), calcium saccharin (sweeteners), sodium saccharin (sweeteners), methyl salicylate (perfumes), calcium oxide (yeast food, reinforcing agents), oxidized starch (thickening stabilizers, manufacturing agents), magnesium oxide (reinforcing agents, manufacturing agents), iron sesquioxide (diiron trioxide) (coloring agents), hypochlorous acid water (disinfectants), sodium hypochlorite (hypochlorite of soda) (disinfectants, bleaching agents), hypobromous acid water (disinfectants), sodium hydrosulfite (hydrosulfite) (preservatives, antioxidants, bleaching agents), 2,3-diethylpyrazine (perfumes), 3-diethyl-5-methylpyrazine (perfumes), allyl cyclohexylpropionate (perfumes), L-cystein monohydrochloride (reinforcing agents), disodium 5′-cytidylate (seasonings), citral (perfumes), citronellal (perfumes), citronellol (perfumes), 1,8-cineole (eucalyptol) (perfumes), diphenyl (biphenyl) (fungicides), butylated hydroxytoluene (antioxidants), dibenzoyl thiamine (reinforcing agents), dibenzoyl thiamine hydrochloride (reinforcing agents), fatty acids (perfumes), aliphatic higher alcohols (perfumes), aliphatic higher aldehydes (perfumes), aliphatic higher hydrocarbons (perfumes), 2,3-dimethylpyrazine (perfumes), 2,5-dimethylpyrazine (perfumes), 2,6-dimethylpyrazine (perfumes), 2,6-dimethylpyridine (perfumes), oxalic acid (manufacturing agents), potassium bromate (manufacturing agents), DL-tartaric acid (dl-tartaric acid) (acidulants), L-tartaric acid (acidulants), potassium hydrogen DL-tartrate (potassium hydrogen dl-tartrate) (seasonings), potassium hydrogen L-tartrate (seasonings), disodium DL-tartrate (disodium dl-tartrate) (acidulants, seasonings), disodium L-tartrate (disodium l-tartrate) (acidulants, seasonings), potassium nitrate (color couplers, manufacturing agents), sodium nitrate (color couplers, manufacturing agents), food red No.2 and its aluminum lake (coloring agents), food red No.3 and its aluminum lake (coloring agents), food red No.40 and its aluminumlake (coloring agents), food red No.102 (coloring agents), food red No.104 (coloring agents), food red No.105 (coloring agents), food red No.106 (coloring agents), food yellow No.4 and its aluminum lake (coloring agents), food yellow No.5 and its aluminum lake (coloring agents), food green No.3 and its aluminumlake (coloring agents), food blue No.1 and its aluminumlake (coloring agents), food blue No.2 and its aluminumlake (coloring agents), sucrose Esters of Fatty Acids (emulsifiers, gum bases, manufacturing agents), silicone resin (manufacturing agents), cinnamyl alcohol (cinnamic alcohols) (perfumes), cinnamaldehyde (cinnamic aldehyde) (perfumes), potassium hydroxide (manufacturing agents), calcium hydroxide (reinforcing agents, manufacturing agents), sodium hydroxide (manufacturing agents), magnesium hydroxide (reinforcing agents, manufacturing agents), sucralose (sweeteners), calcium stearate (reinforcing agents, manufacturing agents), magnesium stearate (manufacturing agents), calcium stearoyl lactylate (emulsifiers), sodium stearoyl lactylate (emulsifiers), sorbitan esters of fatty acids (emulsifiers, gum bases), D-sorbitol (manufacturing agents), sorbic acid (preservatives), potassium sorbate (preservatives), calcium sorbate (preservatives), ammonium carbonate (manufacturing agents), Potassium Carbonate (anhydrous) (manufacturing agents), calcium carbonate (gum bases, reinforcing agents, manufacturing agents), ammonium hydrogencarbonate (manufacturing agents), sodiumhydrogen carbonate (manufacturing agents), sodium carbonate (manufacturing agents), magnesium carbonate (reinforcing agents, manufacturing agents), thiabendazole (fungicides), thiamine hydrochloride (reinforcing agents), thiamine mononitrate (reinforcing agents), thiamine dicetylsufate (reinforcing agents), thiamine thiocyanate (reinforcing agents), thiamine naphthalene-1,5-disulfonate (reinforcing agents), thiamine dilaurylsulfate (reinforcing agents, manufacturing agents), thioethers (perfumes), thiols (thioalcohols) (perfumes), L-theanine (seasonings, reinforcing agents), decanal (perfumes), decanol (perfumes), ethyl decanoate (perfumes), sodium iron chlorophyllin (coloring agents), 5,6,7,8-tetrahydroquinoxaline (perfumes), 2,3,5,6-tetramethylpyrazine (perfumes), sodium dehydroacetate (preservatives), terpineol (perfumes), terpene Hydrocarbons (perfumes), sodium carboxymethylstarch (thickening stabilizers), copper salts (copper gluconate) (cupric sulfate) (reinforcing agents), sodium copper chlorophyllin (coloring agents), copper chlorophyll (coloring agents), dl-a-tocopherol (antioxidants), tocopheryl acetate (reinforcing agents), d-a-tocopheryl acetate (reinforcing agents), DL-tryptophan (seasonings, reinforcing agents), L-tryptophan (seasonings, reinforcing agents), trimethylamine (perfumes), 2,3,5-trimethylpyrazine (perfumes), DL-threonine (seasonings, reinforcing agents), L-threonine (seasonings, reinforcing agents), nisin (preservatives), natamycin (manufacturing agents), sodium methoxide (manufacturing agents), nicotinic acid (reinforcing agents), nicotinamide (reinforcing agents), sulfur dioxide (preservatives, antioxidants, bleaching agents), chlorine dioxide (manufacturing agents), silicon dioxide (manufacturing agents), carbon dioxide (acidulants, manufacturing agents), titanium dioxide (coloring agents), lactic acid (acidulants), potassium lactate (seasonings, pH adjusters), calcium lactate (seasonings, reinforcing agents), iron lactate (reinforcing agents), sodium lactate (acidulants, seasonings), neotame (sweeteners), γ-nonalactone (perfumes), potassium norbixin (coloring agents), sodium norbixin (coloring agents), vanillin (perfumes), isobutyl p-hydroxybenzoate (preservatives), isopropyl p-hydroxybenzoate (preservatives), ethyl p-hydroxybenzoate (preservatives), butyl p-hydroxybenzoate (preservatives), propyl p-hydroxybenzoate (preservatives), p-methylacetophenone (perfumes), L-valine (seasonings, reinforcing agents), valeraldehyde (perfumes), calcium pantothenate (reinforcing agents), sodium pantothenate (reinforcing agents), biotin (reinforcing agents), L-histidine monohydrochloride (seasonings, reinforcing agents), bisbentiamine (reinforcing agents), vitamin A (reinforcing agents), vitamin A fatty acids esters (reinforcing agents), 1-hydroxyethylidene-1,1-diphosphonic acid (disinfectants (component of peracetic acid formulation)), hydroxycitronellal (perfumes), hydroxycitronellal dimethylacetal (perfumes), hydroxypropyl distarch phosphate (thickening stabilizers, manufacturing agents), hydroxypropyl cellulose (manufacturing agents), hydroxypropyl starch (thickening stabilizers, manufacturing agents), hydroxypropyl methylcellulose (manufacturing agents), piperidine (perfumes), piperonal (perfumes), piperonyl butoxide (manufacturing agents), sunflower lecithin (emulsifiers), glacial acetic acid (acidulants, manufacturing agents), pyrazine (perfumes), pyridoxine hydrochloride (reinforcing agents), pyrimethanil (fungicides), potassium pyrosulfite (preservatives, acidulants, bleaching agents), sodium pyrosulfite (preservatives, acidulants, bleaching agents), pyrrolidine (perfumes), tetrapotassium pyrophosphate (manufacturing agents), calcium dihydrogen pyrophosphate (reinforcing agents, manufacturing agents), disodium dihydrogen pyrophosphate (manufacturing agents), ferric pyrophosphate (reinforcing agents), tetrasodium pyrophosphate (manufacturing agents), pyrrole (perfumes), L-phenylalanine (seasonings, reinforcing agents), isoamyl phenylacetate (perfumes), isobutyl phenylacetate (perfumes), ethyl phenylacetate (perfumes), 2-(3-Phenylpropyl)pyridine (perfumes), phenethylamine (perfumes), phenol ethers (perfumes), phenols (perfumes), ferrocyanides (325-1 potassium ferrocyanide, 325-2 calcium ferrocyanide, 325-3 sodium ferrocyanide) (manufacturing agents), butanol (perfumes), butylamine (perfumes), butyraldehyde (perfumes), butylated hydroxyanisole (antioxidants), fumaric acid (acidulants), monosodium fumarate (acidulants, seasonings), fludioxonil (fungicides), furfurals and its derivatives (perfumes), propanol (perfumes), propionaldehyde (perfumes), propionic acid (preservatives, perfumes), isoamyl propionate (perfumes), ethyl propionate (perfumes), calcium propionate (preservatives), sodium propionate (preservatives), benzyl propionate (perfumes), propiconazole (fungicides), propylene glycol (manufacturing agents), propylene glycol esters of fatty acids (emulsifiers, gum bases), hexanoic acid (perfumes), allyl hexanoate (perfumes), ethyl hexanoate (perfumes), ethyl heptanoate (perfumes), 1-perillaldehyde (perfumes), benzyl alcohol (perfumes), benzaldehyde (perfumes), 2-pentanol (another name: sec-amylalcohol) (perfumes), trans-2-pentenal (perfumes), 1-penten-3-ol (perfumes), aromatic alcohols (perfumes), aromatic aldehydes (perfumes), propyl gallate (antioxidants), sodium polyacrylate (thickening stabilizers), polyisobutylene (gum bases), polysorbate 20 (emulsifiers), polysorbate 60 (emulsifiers), polysorbate 65 (emulsifiers), polysorbate 80 (emulsifiers), polyvinylpyrroridone (manufacturing agents), polyvinylpolypyrrolidone (manufacturing agents), polybutene (gum bases), potassium polyphosphate (manufacturing agents), sodium polyphosphate (manufacturing agents), d-borneol (perfumes), maltol (perfumes), D-mannitol (manufacturing agents), potassium metaphosphate (manufacturing agents), sodium metaphosphate (manufacturing agents), DL-methionine (seasonings, reinforcing agents), L-methionine (seasonings, reinforcing agents), methyl N-methylanthranilate (perfumes), 5-methylquinoxaline (perfumes), 6-methylquinoline (perfumes), 5-methyl-6,7-dihydro-5 H-cyclopentapyrazine (perfumes), methyl cellulose (thickening stabilizers), 1-methylnaphthalene (perfumes), methyl β-naphthyl ketone (perfumes), 2-methypyrazine (perfumes), 2-methylbutanol (perfumes), 3-methyl-2-butanol (perfumes), 2-methylbutyraldehyde (perfumes), trans-2-methyl-2-butenal (perfumes), 3-methyl-2-butenal (perfumes), 3-methyl-2-butenol (perfumes), methyl hesperidin (reinforcing agents), dl-menthol (perfumes), l-menthol (perfumes), morpholine salts of fatty acids (manufacturing agents), folic acid (reinforcing agents), butyric acid (perfumes), isoamyl butyrate (perfumes), ethyl butyrate (perfumes), cyclohexyl butyrate (perfumes), butyl butyrate (perfumes), lactones (perfumes), L-lysine L-aspartate (seasonings, reinforcing agents), L-lysine monohydrochloride (seasonings, reinforcing agents), L-lysine L-glutamate (seasonings, reinforcing agents), linalool (perfumes), calcium 5′-ribonucleotide (seasonings), disodium 5′-ribonucleotide (seasonings), riboflavin (coloring agents, reinforcing agents), riboflavin tetrabutyrate (coloring agents, reinforcing agents), riboflavin 5′-phosphate sodium (coloring agents, reinforcing agents), sulfuric acid (manufacturing agents), aluminum ammonium sulfate (raising agents, manufacturing agents), aluminum potassium sulfate (raising agents, manufacturing agents), ammonium sulfate (manufacturing agents), potassium sulfate (seasonings), calcium sulfate (reinforcing agents, coagulants for tofu), ferrous sulfate (reinforcing agents), sodium sulfate (manufacturing agents), magnesium sulfate (manufacturing agents, reinforcing agents), DL-malic acid (dl-malic acid) (acidulants), sodium DL-malate (sodium dl-malate) (acidulants, seasonings), phosphoric acid (acidulants, manufacturing agents), distarch phosphate (thickening stabilizers, manufacturing agents), monostarch phosphate (thickening stabilizers, manufacturing agents), tripotassium phosphate (seasonings, manufacturing agents), tricalcium phosphate (gum bases, reinforcing agents, manufacturing agents), trimagnesium phosphate (reinforcing agents, manufacturing agents), diammonium hydrogen phosphate (manufacturing agents), ammonium dihydrogen phosphate (manufacturing agents), dipotassium hydrogen phosphate (seasonings, manufacturing agents), potassium dihydrogen phosphate (seasonings, manufacturing agents), calcium monohydrogen phosphate (gum bases, reinforcing agents, manufacturing agents), calcium dihydrogen phosphate (reinforcing agents, manufacturing agents), disodium hydrogen phosphate (seasonings, manufacturing agents), sodium dihydrogen phosphate (seasonings, manufacturing agents), magnesium monohydrogen phosphate (reinforcing agents, yeast food), trisodium phosphate (seasonings, manufacturing agents), phosphated distarch phosphate (thickening stabilizers, manufacturing agents).

Use of an additive in the present invention is advantageous for effectively improving the bioabsorbability of a solid carotenoid derived from a genus Paracoccus microorganism. Use of an additive in the composition of the present invention is advantageous for effectively improving the fast-acting property, persistence, and/or water solubility or water dispersibility of a carotenoid. Use of an additive in the composition of the present invention is advantageous for effectively improving the workability, availability, and/or stability of a carotenoid. The workability and availability as used herein mean, for example, that the carotenoid of the present invention is easily formulated (e.g., can be uniformly formulated) into a food and drink such as bread, juice, and pastes, or that the quality such as taste, flavor, color, and texture of a food and drink to which the carotenoid of the present invention is added, is improved, or that the carotenoid of the present invention does not largely affect the quality of a food and drink.

The form of the composition of the present invention is not particularly limited as long as the effect of the present invention is not prevented, and may be solid, semi-solid (including paste and gel), liquid (including oil and slurry), and gas (e.g., aerosol), and the form is preferably solid or liquid. The liquid composition also includes emulsion.

The dosage form of the composition of the present invention is not particularly limited as long as the effect of the present invention is not prevented, and examples thereof include injection, tablet (e.g., plain tablet, sugar coated tablet, film-coated tablet, enteric-coated tablet, controlled-release tablet, orally disintegrating tablet, sublingual tablet, chewable tablet, etc.), capsule (e.g., hard capsule, soft capsule), elixir, pill, dust, powder, granule, beadlet (e.g., carbon dioxide may be included), solution, troche, syrup, dry syrup, emulsion, suspension, liquid, inhalant, aerosol agent, powder inhalant, suppository, ointment, cream, gel, patch, poultice, lotion, drop, ophthalmic ointment, eye drop, and nasal drop. The dosage form of the composition of the present invention is preferably a dosage form for oral intake or administration, and examples thereof include tablet, capsule, pill, dust, powder, granule, syrup, dry syrup, emulsion, liquid, suspension, solution, and troche.

The composition of the present invention can be prepared by a known method such as mixing, dissolving, dispersing, and suspending the above-mentioned solid carotenoid and, if desired, an orally acceptable or pharmaceutically acceptable additive according to the form or dosage form thereof. In preparation of the composition of the present invention, a mixture, a dissolved substance, a dispersed substance, a suspension, etc., prepared by the above-mentioned method may be subjected to homogenization treatment or sterilization treatment, as long as the effect of the present invention is not prevented.

According to one embodiment of the method for producing the composition of the present invention, when the composition of the present invention is a solid (e.g., powdered) composition, a step of grinding the above-mentioned solid carotenoid may be included. Furthermore, a step of mixing an additive such as an emulsifier may be included before and/or after the grinding step. After the step for grinding the solid carotenoid mentioned above, a step of mixing a solvent or dispersion medium may be included if necessary. Furthermore, it is preferable to include a step of removing the solvent or dispersion medium and/or a component such as a liquid in the solid carotenoid from the mixture obtained by the step of mixing a solvent or dispersion medium mentioned above. The grinding method mentioned above is not limited as long as it is a method that can sufficiently grind a solid carotenoid, and examples thereof include a jet mill grind method, a ball mill grinding method, a bead mill grinding method, a roller mill grinding method, a hammer mill grinding method, a pin mill grinding method, and a combination thereof. The mixing method mentioned above is not limited as long as it is a method that can sufficiently mix a solid carotenoid with an additive, and examples thereof include a mixing method with a mortar, a mixing method with a screw, a mixing method with a stirring blade, a mixing method by the effect of centrifugal force and gravity by rotating a container, a mixing method by the effect of a fracturing method, and a combination thereof. The removing method mentioned above is not limited as long as it is a method that can sufficiently dry a solvent and water, and examples thereof include a spray dry method, a freeze-drying method, a vacuum drying method, a drum drying method, a far-infrared drying method, a high-frequency drying (microwave) method, a hot-air drying method, a fluidized bed drying method, a low-temperature drying method, a press drying method, a dielectric heat drying method, and a combination thereof.

According to another embodiment of the method for producing the composition of the present invention, when the composition of the present invention is a liquid (e.g., suspension, emulsion (emulsified liquid), or solution), a step of mixing the above-mentioned solid carotenoid with a solvent or dispersion medium may be included. By such step, it is possible to suspend, emulsify, or dissolve the above-mentioned solid carotenoid. Before and/or after the step of mixing a solvent or dispersion medium, a step of mixing an additive such as an emulsifier may be included, if necessary. When the composition of the present invention is an emulsion, before the step of mixing a dispersion medium, a step of mixing an additive such as a solvent and of dissolving a solid carotenoid may be included, if necessary.

The above-mentioned liquid composition may be dried to make a solid (e.g., powdered) composition.

According to another embodiment of the method for producing the composition of the present invention, a step of mixing the above-mentioned solid carotenoid with a solvent or dispersion medium to dissolve or suspend may be included. Before and/or after the step of mixing a solvent, a step of mixing an additive such as an emulsifier may be included, if necessary. It is preferable to further include a step of removing or reducing the solvent or dispersion medium and/or a component such as a liquid in the solid carotenoid from the mixture obtained by the mixing step mentioned above. The removing or reducing method mentioned above is not limited as long as it is a method that can sufficiently dry a solvent, a dispersion medium, and water, and examples thereof include a spray dry method, a freeze-drying method, a vacuum drying method, a drum drying method, and a far-infrared drying method. Here, by preparing the drying condition, it is possible to prepare the water content and the particle size to make a powdered, granular, or pasty composition. Furthermore, the granular composition thus obtained may be coated with an additive to make a beadlet. By grinding the dried composition, it is possible to obtain a powdered or granular composition.

The lower limit of the particle size of the composition of the present invention is not particularly limited, and may be a volume-based median diameter (D50) of, for example, 10 nm or more, preferably 100 nm or more, and more preferably 300 nm or more. The upper limit of the particle size of the composition is a volume-based median diameter of, for example, 1000 μm or less, preferably 700 μm or less, and more preferably 500 μm or less. Therefore, the composition of the present invention may be a nanoparticle.

The particle size distribution of the composition can be measured using a particle size distribution measuring device utilizing a laser diffraction scattering method. Specifically, the measurement can be easily performed by using a commercial device (e.g., LMS-2000e, Seishin Enterprise Co., Ltd.).

A method for administration or intake of the composition of the present invention is not particularly limited, and examples thereof include injection such as infusion, intravenous injection, intramuscular injection, subcutaneous injection, and intradermal injection, and oral, transmucosal, percutaneous, intranasal, intraoral, etc., administration or intake, and the method is preferably oral intake or administration.

Examples of the composition of the present invention include foods and drinks such as foods and drinks, food additives, feeds, pharmaceuticals, quasi drugs, or cosmetics, or materials thereof, and foods and drinks or food additives or materials thereof are preferred in terms of simpleness of intake.

Examples of the food and drink of the present invention include the composition of the present invention prepared as a food and drink as it is (e.g., the dosage form of the composition of the present invention is prepared as a food and drink as it is). When the composition of the present invention is used as a food and drink material, various proteins, saccharides, fats, trace elements, vitamins, plant extracts, or other active ingredients (e.g., bacteria such as lactic acid bacteria and Bacillus bacteria, fungi such as yeasts, dietary fibers, DHA or EPA) may be further formulated into the composition of the present invention to make a food and drink, the composition of the present invention may be made into a liquid (such as solution), semiliquid, or solid food and drink, or the composition of the present invention may be added to a general food and drink to make a food and drink.

Specific examples of the above-mentioned food and drink include instant foods such as instant noodles, pre-packaged foods, canned foods, foods for microwave cooking, instant soups and miso soups, and freeze-dried foods; drinks such as soft drinks, fruit juice drinks, vegetable drinks, soy milk drinks, coffee drinks, tea drinks, powdered drinks, concentrated drinks, and alcoholic drinks; energy drinks; flour products such as breads, pastas, noodles, cake mixes, and bread crumbs; confectionery such as candies, gummies, jellies, caramels, chewing gums, chocolates, cookies, biscuits, cakes, pies, snacks, crackers, Japanese-style confectionery, and dessert confectionery; nutrition bars; seasonings such as sauces, processed tomato seasonings, flavor seasonings, cooking mixes, sauces, dressings, soups, and curry or stew mixes; oils and fats such as processed oils and fats, butter, margarine, and mayonnaise; dairy products such as milk-based drinks, yogurts, lactic acid bacteria drinks, ice creams, and creams; processed agricultural products such as agricultural canned foods, jams and marmalades, and cereals; processed meat foods such as hams, bacons, sausages, and roast pork: and frozen foods, but the food and drink is not limited thereto.

The food and drink of the present invention also include health foods, supplements, functional foods (e.g., including foods for specified health uses, functional nutritional foods, or foods with function claims), foods for special dietary uses (e.g., including foods for the sick, infant formulas, powdered milk for pregnant and lactating women, or foods for persons with swallowing/chewing difficulties), or liquid modified milk for infants (also referred to as liquid milk for infants).

The intake or dose of the composition of the present invention is not particularly limited, and can be determined depending on the prescription of the composition, the type of a carotenoid, purity, the type of a subject, age or body weight of a subject, symptoms, the duration of intake or administration, the form of the composition, the method for intake or administration, a combination of a carotenoid other than the carotenoid of the present invention or a drug, and the like. The composition of the present invention is preferably composed of a form of daily intake unit so that the effective dose is for inhibition or treatment of a desired disease or a symptom attributable thereto. For example, when the composition of the present invention is orally taken, the carotenoid can be formulated into the composition so that the intake or dose of the carotenoid is in the range of 0.001 to 100 mg, preferably 0.005 to 70 mg, and more preferably 0.01 to 50 mg per day per adult with a body weight of 60 kg. The intake or dose of a carotenoid other than the carotenoid of the present invention or a drug used in combination with the carotenoid of the present invention can also be appropriately determined using a clinically used intake or dose, respectively, as a standard.

“Inhibition” of a disease or a symptom attributable thereto as used herein includes the meaning of improvement in a disease or a symptom attributable thereto by a non-medical practice, as well as the meaning of “prevention” in which provision is made for expected worsening in advance and occurrence or recurrence of a disease or a symptom attributable thereto is prevented in the bud by a non-medical practice or a medical practice. “Treatment” means improvement in a disease or a symptom attributable thereto by a medical practice. Improvement as used herein includes stopping, alleviating, or delaying the progress or worsening of a disease or a symptom attributable thereto.

The daily intake or dose of the composition of the present invention is appropriately selected according to the prescription of the composition, etc., like the intake or dose of the composition mentioned above. The daily intake or dose of the composition of the present invention may be, for example, taken by or administered to a subject once or plural times, and is preferably taken by or administered to a subject once. Therefore, the daily number of intake or administration of the composition of the present invention is 1 to 5 times a day, preferably 1 to 3 times a day, and more preferably once a day.

According to one embodiment of the present invention, a subject to whom the composition of the present invention is applied is not particularly limited as long as the effect of the present invention is not prevented, and is preferably mammals, and more preferably primates such as humans, dogs, and cats. The subject may be healthy subjects (healthy animals) or patients (patient animals).

The composition of the present invention is suitable for addition to pharmaceuticals or foods and drinks, etc., of various dosage forms or forms such as drinks, soft capsules, gummies, jellies, and breads. Therefore, according to the composition of the present invention, it is advantageous for effectively improving the workability and/or availability of a solid carotenoid.

Furthermore, the composition of the present invention can also be used for changing in the properties (e.g., texture, taste, odor, color, etc.) of foods and drinks since the composition improves the workability, availability, water solubility, or water dispersibility, etc., of a solid carotenoid.

As the preferable first embodiment of the composition of the present invention, a composition comprising an additive such as a solvent (e.g., a lipophilic liquid) together with a solid carotenoid is exemplified.

As the method for producing the composition in the preferable first embodiment mentioned above, a step of mixing the above-mentioned solid carotenoid with a solvent (e.g., a lipophilic liquid) is included. Here, the mixing can be performed in other additives (e.g., surfactants, solvents, dispersion media, antioxidants, and excipients, etc.). According to another first embodiment of the present invention, after the mixing step, the mixture thus obtained may be dispersed in other additives (e.g., surfactants, solvents, dispersion media, antioxidants, and excipients, etc.), if desired. Examples of the solvent or dispersion medium include water, lower alcohol, polyhydric alcohol, and a mixture of two or more thereof.

As the preferable second embodiment of the composition of the present invention, a solid composition (e.g., a powdered composition) comprising an additive such as fat-soluble vitamins together with a solid carotenoid is exemplified.

As the method for producing the composition in the preferable second embodiment mentioned above, a step of grinding a solid carotenoid is included. Before and/or after the grinding step, a step of mixing an additive (e.g., fat-soluble vitamins) may be included.

As the preferable third embodiment of the composition of the present invention, a solid composition (e.g., a powdered composition) or a liquid composition (e.g., a colloid solution or an aqueous emulsion (an oil-in-water dispersed substance) of the powdered composition) comprising an additive such as thickeners (e.g., pectin), oily solvents (e.g., soybean oil such as partially hydrogenated soybean oil), dispersion media (e.g., water), and if necessary, excipients, antioxidants, and the like together with a solid carotenoid is exemplified.

In the method for producing the composition in the preferable third embodiment mentioned above, a step of dissolving a thickener such as sugar beet pectin, and if necessary, an excipient such as sugar in water to obtain an aqueous pectin solution is included. Furthermore, a step of dissolving and/or suspending a solid carotenoid and, if necessary, an additive such as an antioxidant in an oily solvent such as soybean oil to obtain a solid carotenoid solution (or a dispersed substance) is included. Thereafter, the solid carotenoid solution (or the dispersed substance) is added to the aqueous pectin solution, and the mixture thus obtained is homogenized to obtain an oil-in-water dispersed substance. Thereafter, the oil-in-water dispersed substance thus obtained is, if necessary, dried to obtain a powdered composition. Here, in the above-mentioned homogenization, for example, a conventional technique such as high-pressure homogenization and high shear emulsification (rotor-stator system) can be used.

As the preferable fourth embodiment of the composition of the present invention, a solid composition (e.g., a beadlet) that includes an additive such as emulsifiers (e.g., gelatin), and, if necessary, excipients (e.g., sugar), and humectants (e.g., glycerin) together with a solid carotenoid and that is coated with a manufacturing agent (e.g., calcium silicate) is exemplified.

In the method for producing the composition in the preferable fourth embodiment mentioned above, a step of mixing an emulsifier (e.g., gelatin) with a humectant (e.g., glycerin) in advance, followed by heating to dissolve in water is included.

Here, an excipient (e.g., sugar) may be added together with the emulsifier. Thereafter, a step of adding a solid carotenoid to perform preliminary emulsification is included. Preliminary emulsification may be held, and then the emulsion may be passed through a high-pressure homogenizer to be converted into a fine droplet. Thereafter, the emulsion droplet thus obtained is sprayed in an agitated cloud or suspension in the air of a particle of a manufacturing agent (e.g., calcium silicate) as a fine disperse powder to obtain a bead let. Thereafter, heat treatment may be performed. Here, in the above-mentioned heat treatment, for example, a conventional technique such as a fluidized bed method can be used.

As the preferable fifth embodiment of the composition of the present invention, a liquid composition (e.g., an aqueous emulsion (an oil-in-water dispersed substance)) including an additive such as emulsifiers (e.g., whey protein, sucrose fatty acid ester, or a combination thereof), solvents (e.g., oily solvents such as MCT), dispersion media (e.g., water), and antioxidants together with a solid carotenoid is exemplified.

As the method for producing the composition in the preferable fifth embodiment mentioned above, the following method is exemplified.

An emulsion can be, for example, prepared by the following two steps:
a preliminary emulsification step for preparation of a crude emulsion by emulsifying a solid carotenoid dissolved in a solvent into a dispersion medium, and
a final emulsify step for preparation of a final emulsion.

As the preferable sixth embodiment of the composition of the present invention, a powdered composition (e.g., a nanoparticle) including emulsifiers (e.g., modified starch), excipients (e.g., sugar), and antioxidants together with a solid carotenoid is exemplified.

As the method for producing the composition in the preferable sixth embodiment mentioned above, the following method is exemplified.
a) a step of dissolving a solid carotenoid at a temperature higher than 30° C. in a solvent (e.g., alcohol) or a mixture of water and a solvent (e.g., alcohol),
b) a step of mixing the solution obtained in a) with a molecular disperse solution or colloidal disperse solution in which excipients (e.g., sugar) and emulsifiers (e.g., modified starch) are mixed, wherein the hydrophobic phase of a solid carotenoid is formed as a nanodisperse phase, and
c) a step of separating the majority of water and a solvent that was additionally used as needed, followed by drying of the disperse liquid thus produced to make a dry powder.

As the preferable seventh embodiment of the composition of the present invention, a powdered composition comprising manufacturing agents (e.g., cyclodextrin, water-soluble cyclodextrin derivatives) together with a solid carotenoid is exemplified.

The method for producing the composition in the preferable seventh embodiment mentioned above is not particularly limited as long as it is a method for clathration, and examples thereof include a kneading method, a liquid phase mixing method, a solvent evaporation method, a coprecipitation method, and a freeze-drying method.

The kneading method is a method in which, for example, using a kneader, a mortar, and the like, proper amounts of water and a solvent (e.g., alcohol, halogenated carbon, or a combination thereof) are added to a mixture of a solid carotenoid and a manufacturing agent (e.g., cyclodextrin, water-soluble cyclodextrin derivatives), and are kneaded until they become pasty while triturating. By drying the pasty composition thus obtained, a powdered composition can be obtained.

As the preferable eighth embodiment of the composition of the present invention, a liquid composition (e.g., a suspension) including an additive such as dispersion media (e.g., ω-3 fatty acid, vegetable oils and fats) together with a solid carotenoid is exemplified.

As the method for producing the composition in the preferable eighth embodiment mentioned above, a step of mixing a solid carotenoid with an additive (e.g., dispersion media) is included. Before the mixing step, a step of grinding a solid carotenoid may be included.

According to another embodiment of the present invention, a method for inhibiting or treating a desired disease or a symptom attributable thereto in a subject, which comprises administration or intake of a carotenoid-containing composition obtained by dispersing an effective dose of a solid carotenoid derived from a genus Paracoccus microorganism to or by the subject, is provided. “Effective dose” as used herein can be set in the same manner as the content of a solid carotenoid, etc., in daily intake unit. The above-mentioned method can also be applied to a subject only by a non-medical practice. Therefore, according to another embodiment of the present invention, a method for inhibiting a disease or a symptom attributable thereto in a subject (excluding a medical practice), which comprises administration or intake of a carotenoid-containing composition obtained by dissolving or dispersing an effective dose of a solid carotenoid derived from a genus Paracoccus microorganism to or by the subject, is provided. The above-mentioned method of the present invention can be performed in accordance with the content mentioned herein in the composition of the present invention.

According to another embodiment of the present invention, a method for improving an absorbability of a carotenoid, comprising dissolving or dispersing a solid carotenoid derived from a genus Paracoccus microorganism in the presence of an orally acceptable or pharmaceutically acceptable excipient.

The embodiment of the improvement method mentioned above can be performed in accordance with the description on the composition of the present invention.

### EXAMPLES

The present invention will be more specifically described below by way of Preparation Examples, Examples, and Test Examples, but the technical scope of the present invention is not limited to these Examples. Unless otherwise specified, all percentages and ratios used in the present invention are by mass. Unless otherwise specified, the unit and the measurement methods as used herein are in accordance with the JIS Standard.

Determination of a carotenoid such as astaxanthin, measurement of particle size distribution, evaluation of absorbability, evaluation of stability, and evaluation of water solubility or water dispersibility in Preparation Examples, Examples, and Test Examples below were performed as follows.

I. Method for Determining Carotenoid such as Astaxanthin

Determination of a carotenoid such as astaxanthin was performed in accordance with the method mentioned in WO 2014/054669 A. The method will be briefly mentioned below.

Determination of a carotenoid such as astaxanthin was performed using high performance liquid chromatography (HPLC). As the column, Wakosil-II SIL-100 (φ4.6×250 mm) (manufactured by Wako Pure Chemical Industries, Ltd.) was used by linking two thereof. Elution was performed by flowing an n-hexane/tetrahydrofuran/nnethanol mixture (volume ratio of 40:20:1), which was a mobile phase, at a flow rate of 1.0 mL/min at a constant temperature near room temperature. In measurement, a solution obtained by dissolving a sample in tetrahydrofuran was diluted 100 times with the mobile phase to obtain 20 μL of a solution, and this was used as an infusion volume, and detection of the carotenoid in the column eluate was performed at a wavelength of 470 nm. As the reference standard for determination, astaxanthin (Cat. No. A9335) manufactured by Sigma was used. The astaxanthin concentration of the standard solution was set by using the following formula after measurement of the area percentage (B) of the peak of astaxanthin when HPLC analysis was performed at an absorbance (A) of 477 nm of the standard solution and under the above-mentioned conditions:

Astaxanthin concentration (mg/L)=A/2150×B×100 (1)

### (Method for Preparing Sample)

As tetrahydrofuran, a product containing 250 ppm of butylhydroxytoluene, which is an antioxidant, is used. Into a 50 mL glass centrifuge tube, 20 to 200 mg of a sample was accurately weighed, and then 150 μL of distilled water was added. To this, 15 mL of tetrahydrofuran/methanol=20/1 was added, followed by mixing well for 5 minutes, and then 30 mL of hexane was added. From this solution, an insoluble matter was precipitated by centrifugation, and supernatant was diluted 1 to 100 times with the mobile phase of HPLC, followed by HPLC analysis.

### (Calculation Method)

The astaxanthin concentration was calculated in accordance with formula (2), and the concentration of other carotenoids was calculated in accordance with formula (3):

Astaxanthin (mg/g)=(astaxanthin concentration (mg/L) in standard solution)/(peak area of astaxanthin standard solution)×(peak area of astaxanthin in sample)/(sample mass (mg))×450 (2)

The concentration of other carotenoids was calculated in accordance with formula (3):

Other carotenoids*¹ (mg/g)=(astaxanthin concentration (mg/L) in standard solution)/(peak area of astaxanthin standard solution)×(peak area*² of target carotenoid in sample)/(sample mass (mg))×450 (3)
(*1: Other carotenoids means adonirubin and adonixanthin)
(*2: Regarding the peak of other carotenoids, the elution time of other carotenoids was measured using a reference standard of the carotenoid in advance)

### II. Measurement of Particle Size Distribution

The particle size distribution of a sample was measured using a laser diffraction and scattering particle size distribution measurement device LMS-2000e (Seishin Enterprise Co., Ltd.). Specifically, the sample was dispersed in water as a dispersion medium and treated by ultrasound for 10 seconds, followed by measurement, and the volume-based median diameter (D50) was recorded.

### III. Evaluation of Absorbability

Single oral administration of 10 mg/kg/day of astaxanthin was performed to rats, and serum was collected 1, 4, and 8 hours after administration. As rats, SD male rats aged 6 weeks at the initiation of the test (5 weeks at the time of obtainment) (SPF) (Japan SLC, Inc.) were used. For rats, general signs were observed at the time of arrival, individuals without abnormality were acclimated for about 1 week from the arrival date, and then healthy individuals without abnormality were subjected to the test. The rats were divided into 5 animals each in 1 group by a body weight stratified randomization method so that the mean body weight in each group was as uniform as possible in the test. Each of rats was maintained in each plastic cage for rats (W27×D44×H19 cm) with bedding in an animal room at room temperature of 20 to 26° C., with a relative humidity of 30 to 70%, and on a 12-h light/dark cycle (lighting time: from 7:00 a.m. to 7:00 p.m.). As feed, solid feed CE-2 (Feedone Co., Ltd.) was available ad libitum. As drinking water, ground water that was sterilized by adding sodium hypochlorite with a facility water supply sterilization device (MJ25SR, Kawamoto Pump Mfg. Co., Ltd.) so that residual chlorine was 0.5 mg/L was filtered through a 5 μm filter, and then it was available ad libitum by a water bottle.

### (Method for Preparing Dosing Solution)

A necessary amount of the test substance was weighed and was suspended in water so that the concentration was a predetermined concentration, and this was used as a dosing solution. The prepared dosing solution was stored under refrigeration and protected from light until use. A specified amount of the dosing solution was weighed, suspended in water for injection by vortexing and mixing by inversion, and then administered to rats. Administration was performed at a dose of 10 mL/kg with a final astaxanthin concentration of 1 mg/mL.

### (Administration Method)

Administration was performed by gavage administration. Specifically, forced intragastric administration was performed using a disposable stomach tube (Fuchigami Kikai Co., Ltd.) and a disposable syringe (Terumo Corporation).

### (Blood Collection)

Blood was collected under isoflurane anesthesia (isoflurane for animals, Mylan N.V.) on the day of administration. Blood was collected 1, 4, and 8 hours after administration. From the blood thus obtained, serum was collected by centrifugation, and then frozen-stored at −40° C.

### (Method for Measuring Serum Carotenoid)

To 1 mL of serum obtained above, 2 mL of ethanol was added, and then 5 mL of a diethyl ether: hexane (2:8, v/v) solution was added, followed by stirring. After allowing to stand, the upper layer was filtered through a filter, followed by evaporation to dryness. The residue was dissolved in an acetone:hexane (2:8,v/v) solution, and subjected to HPLC. Regarding organs, organs were homogenized, and extraction was repeated with acetone until no color occurred. Then, filtration through a filter was performed to evaporate acetone, and diethyl ether; hexane (2:8,v/v) was added to the solution to extract a carotenoid. Furthermore, evaporation to dryness was performed, and the residue was dissolved in acetone:hexane (2:8,v/v) and subjected to HPLC. As the HPLC device, a Hitachi L-6000 intelligent pump and an L-4250 UV-VIS detector were used. The measurement wavelength was 450 nm, and a column of 5 μm Cosmosil 5SL-II (inner diameter of 250×4.6 mm) (Nacalai Tesque, Inc., Japan) was used. As the solvent, acetone:hexane (2:8,v/v) was used, and measurement was performed at a flow rate of 1.0 mL/min.

As evaluation of fast-acting property, (serum astaxanthin concentration 1 hour after administration/serum astaxanthin concentration 4 hours after administration)×100 was defined as fast-acting property %. Of the fast-acting property % obtained, less than 60% was regarded as “×”, 60% or more was regarded as “Δ”, 70% or more was regarded as “◯”, and 80% or more was regarded as “⊚”. “Δ” or more is preferable.

As evaluation of persistence, (serum astaxanthin concentration 8 hours after administration/serum astaxanthin concentration 4 hours after administration)×100 was defined as persistence %. Of the persistence % obtained, less than 60% was regarded as “×”, 60% or more was regarded as “Δ”, 70% or more was regarded as “◯”, and 80% or more was regarded as “⊚”. “Δ” or more is preferable.

### IV. Evaluation of Stability

Ten grams (10 g) each of a dried crystal obtained in Preparation Example 1 mentioned later and carotenoid-containing compositions 1 to 7 was put in an aluminum bag, air was pushed out as much as possible, and the bag was sealed. After storage at room temperature for 3 months, astaxanthin was measured by the method mentioned in the “method for determining carotenoid such as astaxanthin” mentioned above, and maintenance of 100 to 80% compared with the initial state was evaluated as “⊚”, maintenance of 79 to 60% compared with the initial state was evaluated as “◯”, maintenance of 59 to 40% compared with the initial state was evaluated as “Δ”, and less than 40% was evaluated as “×”. Considering further processing, “Δ” or more was judged to be preferable.

### V. Evaluation of Water Solubility or Water Dispersibility

Each of carotenoid-containing compositions 2, 4, 5, and 6, and a dried crystal obtained in Preparation Example 1 was added to 50 mL of water so that astaxanthin was 1 mg, and ultrasonic vibration was performed for 5 minutes, followed by centrifugation, and then the content of astaxanthin in the supernatant fluid was measured.

Evaluation of water solubility was visually performed under light with an illuminance of 1500 lux by a panel having healthy visual acuity (visual acuity of 0.7 or more) for an aqueous solution containing carotenoid-containing composition 6 after centrifugation mentioned above. Evaluation was performed by 10 panels. As a result, “⊚” represents a state of no precipitation, “◯” represents a state of an extremely trace amount of precipitation, “Δ” represents a state of a trace amount of precipitation, and “×” represents a state of complete precipitation of the added composition. In terms of water solubility, “Δ” or more is preferable.

Evaluation of water dispersibility was visually performed under light with an illuminance of 1500 lux by a panel having healthy visual acuity (visual acuity of 0.7 or more) after each of carotenoid-containing composition 2, 4, and 5, and a dried crystal obtained in Preparation Example 1 was added to 50 mL of water so that astaxanthin was 1 mg, followed by vortexing and allowing to stand for 24 hours. Evaluation was performed by 10 panels. As a result, “⊚” represents a state in which all of the above-mentioned compositions or dried crystal (hereinafter referred to as compositions, etc.) are uniformly dispersed in water, “◯” represents a state in which the above-mentioned compositions, etc., are uniformly dispersed in water but an extremely trace amount of compositions, etc., are aggregated, separated, and/or precipitated, “Δ” represents a state in which the above-mentioned compositions, etc., are uniformly dispersed in water but a trace amount of compositions, etc., are aggregated, separated, and/or precipitated, and “×” represents a state in which the added compositions, etc., are completely aggregated, separated, and/or precipitated. In terms of water dispersibility, “Δ” or more is preferable.

### Preparation Example 1

### Preparation of Solid Carotenoid (Dried Crystal)

A solid carotenoid was prepared in accordance with the method mentioned in Example 2 of WO 2014/054669 A. The method will be briefly mentioned below.

### Step 1: Step of Culturing E-396 Strain

An E-396 strain (FERM BP-4283) was cultured in accordance with the method mentioned in Example 1 of JP 2007-319015 A, a dried bacterial cell containing a carotenoid containing about 17 mg of astaxanthin in 1 g as a culture was obtained.

### Step 2: Step of Extracting Ethanol

To 25 kg of the dried bacterial cell obtained in step 1 of this Preparation Example, 550 kg of ethanol was added, and a carotenoid containing astaxanthin was extracted while stirring for 15 minutes at 90° C. under nitrogen atmosphere in a high-pressure container. After the solution temperature was cooled to 65° C., the pressure container was opened, the bacterial cell was removed from the extract by filtration, and further the bacterial cell cake was washed with ethanol to obtain 550 kg of an extract with astaxanthin of 0.07% (wt/wt) and a carotenoid weight concentration of 0.1% (wt/wt).

### Step 3: Step of Concentration, Heat Treatment (Incubation), and Crystallization of Extract

Regarding the extract obtained in step 2 of this Preparation Example, the reduced pressure was adjusted so that the can temperature was 30° C., part of ethanol was distilled away using a rotary evaporator, and the solid concentration was concentrated to a concentration 5-fold higher than the extract (an astaxanthin weight concentration of 0.35% (wt/wt), a carotenoid weight concentration of 0.50% (wt/wt)). Then, this concentrated solution was subjected to heat (incubation) treatment under nitrogen atmosphere at 60° C. for 4 hours, followed by maturing overnight at a can temperature of 30° C. to precipitate a crystal.

### Step 4: Step of Collection by Filtration, Washing, and Drying of Crystal

From a solution containing the crystal obtained in step 3 of this Preparation Example, the crystal was recovered by filtration. This crystal was dried under reduced pressure at 100° C. for 2 hours to obtain 425 g of a dried crystal.

Steps 1 to 4 were repeated 50 times to obtain a total of 22 kg of dried crystals, and they were mixed. The carotenoid content in this mixed dried crystal was determined by the “method for determining carotenoid such as astaxanthin” mentioned above. As a result, the carotenoid content included an astaxanthin content of 60%, an adonirubin content of 11%, and adonixanthin of 7%. The particle size distribution of the dried crystal obtained in this Preparation Example was measured, and the result showed D50 of 50 μm.

### Example 1

### Preparation of Carotenoid-Containing Composition 1

The dried crystal obtained in Preparation Example 1 was ground and mixed to obtain carotenoid-containing composition 1. Specifically, using a jet mill FS-4 (Seishin Enterprise Co., Ltd.), the dried crystal was ground at a throughput of 1 kg/hr and pressure of 0.7 MPa. As a result, the D50 of the dried crystal decreased to 5 μm. Then, 30 g of the ground dried crystal and 100 g of vitamin premix type RD-V (DSM) were mixed at a speed of 300 for 1 hour using a magnet mortar and a stirrer for magnet mortar MMPS-T1 (AS ONE Corporation) to obtain carotenoid-containing composition 1.

The composition of the vitamin premix type RD-V was as follows.

| | | |
|---|---|---|
| Vitamin A | 16,500 | IU/g |
| Vitamin D3 | 1,000 | IU/g |
| Extracted tocopherol | 50.0 | mg/g |
| Dibenzoyl thiamine hydrochloride | 13.0 | mg/g |
| (as thiamine hydrochloride | 7.5 | mg/g) |
| Pyridoxine hydrochloride | 12.2 | mg/g |
| Cyanocobalamin | 10.0 | μg/g |
| Nicotinamide | 95.0 | mg/g |
| Calcium pantothenate | 38.2 | mg/g |
| Folic acid | 1.0 | mg/g |
| L-ascorbic acid | 300.0 | mg/g |
| Other food materials | | |

The results of carotenoid analysis showed that astaxanthin was 13%, adonirubin was 2.5%, and adonixanthin was 1.5%. Carotenoid-containing composition 1 was suitable for addition to tablets, capsules, nutrition bars, breads, and the like. Therefore, such composition is considered to have improved workability and availability.

### Example 2

### Preparation of Carotenoid-Containing Composition 2 (Carotenoid-Containing Powder)

A dried premix of 80 g of pectin (GENU Pectin Type Beta of Copenhagen Pectin A/S; viscosity of a 10% aqueous solution of pectin is about 4000 mPa·s at 50° C.), 160 g of sucrose, and 80 g of maltodextrin (DE of 20 to 23) was prepared. The dried premix was dissolved in 1200 ml of deionized water at 60° C., and 335 g of additional maltodextrin (DE of 20 to 23) was added. After the solid was completely dissolved, 8.0 g of sodium ascorbate was added to make a mixture (=solution A).

One hundred thirty-six grams (136 g) of triglyceride (partially hydrogenated soybean oil of Durkex 500, Loders Croklaan B.V.; AA Wormerveer The Netherlands) and 0.9 g of dl-α-tocopherol were mixed and heated to 140° C. Subsequently, 11 g of the dried crystal obtained in Preparation Example 1 was suspended in a mixture of triglyceride and tocopherol. By stirring at 140° C. for about 10 minutes, a transparent solution of a carotenoid (=solution B) was obtained.

After solution A was heated to 70° C., 135 g of solution B was added to solution A while gently stirring to prepare a crude emulsion. By passing the crude emulsion through high-pressure homogenization treatment (desktop high-pressure homogenizer Panda PLUS 2000, manufactured by Niro Soavi) at 50/300 bar pressure 5 times, a fine emulsion was obtained. By adding the same volume of deionized water at 60° C., the fine emulsion was diluted, and then spray drying was performed at an inlet temperature of 200 to 210° C. and an outlet temperature of 70 to 75° C. using a laboratory spray dryer (ADL-311SA, Yamato Scientific Co., Ltd.). The spray-dried powder thus obtained was dried in a vacuum oven at room temperature overnight to obtain carotenoid-containing composition 2.

The water content of carotenoid-containing composition 2 was 2.4%. The results of carotenoid analysis of the powder showed that astaxanthin was 0.6%, adonirubin was 0.1%, and adonixanthin was 0.06%. Carotenoid-containing composition 2 was suitable for addition to tablets, capsules, gummies, jellies, breads, and the like. Therefore, such composition is considered to have improved workability and availability.

### Example 3

### Preparation of Carotenoid-Containing Composition 3 (Carotenoid-Containing Beadlet)

Ninety grams (90 g) of gelatin bloom 140 and 18 g of fructose were added to 313.2 g of water (containing 23.2 g of glycerin) and heated to 65° C. to be dissolved. Then, 158 g of the ground dried crystal obtained in Example 1 was mixed with the matrix thus obtained, followed by preliminary emulsification. Then, the emulsion was passed through a high-pressure homogenizer to make a fine droplet. Then, by blowing calcium silicate as a fine disperse powder, the above-mentioned droplet was solidified to make a beadlet. The average particle size of the beadlet was in the range of 200 to 300 μm. Then, treatment was performed with a fluidized bed. In the fluidized bed, the temperature was controlled to 100 to 115° C. for 5 minutes.

Regarding the carotenoid content in carotenoid-containing composition 3 thus obtained, astaxanthin was 30%, adonirubin was 5.5%, and adonixanthin was 3.5%. Carotenoid-containing composition 3 was suitable for addition to tablets, capsules, breads, and the like. Therefore, such composition is considered to have improved workability and availability.

### Example 4

### Preparation of Carotenoid-Containing Composition 4 (Carotenoid-Containing O/W Emulsion)

A carotenoid-containing O/W emulsion having the following composition was prepared.

The composition of the O/W emulsion (per 1 kg of the emulsion) was as follows:
Continuous phase (60% by weight of the emulsion)

578.3 g of redistilled water

10 g of sucrose laurate (L-1695, manufactured by Mitsubishi-Kagaku Foods Corporation)

10 g of whey protein (BiPRO (registered trademark), manufactured by Davisco Foods International, Inc., U.S.A)
Dispersed phase (40% by weight of the emulsion)

0.7 g of α-tocopherol

3 g of the dried crystal obtained in Preparation Example 1

398 g of medium-chain triglyceride (Miglyol)

This emulsion was prepared as follows. The dried crystal obtained in Preparation Example 1 was suspended in medium-chain triglyceride, and ground with a vibration mill to make a particle size of about 20 μm. The finely ground suspension was sent to a heated coil, and the temperature was temporarily raised to 160 to 200° C. At this time, the carotenoid was dissolved (hereinafter also referred to as dispersed phase (α)). Next, the dispersed phase (α) was mixed with an emulsifier system (γ) (sucrose laurate and whey protein) and α-tocopherol, and this was added together with a dispersion medium (β) (redistilled water) and homogenized at 10,000 rotations/min for about 15 seconds using a homogenizer Ultra Turrax (Yamato Scientific Co., Ltd.). The temperature of the emulsion was about 70° C. Then, finally, this crude emulsion was passed through a slit at 600 bar to be emulsified, and subsequently further emulsified at 1,000 bar using Microfluidizer (Microfluidics). This procedure was repeated 3 times in order to obtain a droplet with a narrow particle size distribution and a small dispersed phase (α), thus obtaining an emulsified liquid (carotenoid-containing composition 4).

Regarding the carotenoid content in carotenoid-containing composition 4, astaxanthin was 0.18%, adonirubin was 0.033%, and adonixanthin was 0.021%. Carotenoid-containing composition 4 was suitable for addition to drinks, soft capsules, gummies, jellies, breads, and the like. Therefore, such composition is considered to have improved workability and availability.

### Example 5

### Preparation of Carotenoid-Containing Composition 5 (Carotenoid-Containing Powdered Composition)

In a solution of 1.7 g of atocopherol and 130 g of isopropanol, 10 g of the dried crystal obtained in Preparation Example 1 was suspended. This suspension was mixed with 460 g of hot isopropanol under a high pressure of 50 bar to obtain a solution at 170° C. This solution was mixed by stirring in a mixing chamber with a solution obtained by dissolving 70.4 g of Purity Gum 2000 (National Starch) and 80.4 g of glucose in 6,450 g of water. Subsequently, isopropanol was removed from this carotenoid disperse liquid with a vacuum evaporator, and the disperse liquid was made into a powder by spray drying.

Regarding the carotenoid content in carotenoid-containing composition 5 (powdered composition), astaxanthin was 6%, adonirubin was 1.1%, and adonixanthin was 0.7%, and the D50 of carotenoid-containing composition 5 was 310 nm. Carotenoid-containing composition 5 was suitable for addition to drinks, soft capsules, gummies, jellies, breads, and the like. Therefore, such composition is considered to have improved workability and availability.

### Example 6

### Preparation of Carotenoid-Containing Composition 6 (Carotenoid-Containing Powdered Composition)

One gram (1.0 g) of the dried crystal obtained in Preparation Example 1, 4.0 g of powdered y-cyclodextrin (trade name: Dexy pearl γ-100, manufactured by Ensuiko Sugar Refining Co., Ltd.), 4 ml of an ethanol-dichloromethane (volume ratio=1:1) mixed solution, and 4 ml of ion exchanged water were put into a mortar, and mixed well with a pestle. After the organic solvent was volatilized, 4 ml of an ethanol-dichloromethane (volume ratio=1:1) mixed solution was added again, followed by mixing well again, and this operation was repeated 3 times to make pasty. Almost all of the solvents added at the final step were volatilized, and the residue of the wet powder was dried overnight under reduced pressure with a vacuum dryer to obtain a powdered composition (carotenoid-containing composition 6).

As a result of analysis of the carotenoids in carotenoid-containing composition 6 (powdered composition), astaxanthin was 12%, adonirubin was 2.2%, and adonixanthin was 1.4%.

### Example 7

### Preparation of Carotenoid-Containing Composition 7 (Carotenoid Oil Suspension)

Thirty grams (30 g) of the ground dried crystal obtained in Example 1 (D50=5 μm) and 100 g of OMEGAVIE 3030 TG QualitySliver Ice (EPA30%/DHA30%) (Polaris, France), which is a DHA/EPA-containing purified fish oil, were mixed with a magnetic stirrer for 1 hour to obtain a carotenoid oil suspension (carotenoid-containing composition 7).

As a result of analysis of the carotenoids in carotenoid-containing composition 7, astaxanthin was 12.5%, adonirubin was 2.4%, and adonixanthin was 1.4%. Carotenoid-containing composition 7 was suitable for addition to soft capsules, sports bars, breads, and the like. Therefore, such composition is considered to have improved workability and availability.

### Test Example 1

### Evaluation of Carotenoid-Containing Composition

Regarding carotenoid-containing compositions 1 to 7 obtained in Examples 1 to 7 and the dried crystal obtained in Preparation Example 1, determination of a carotenoid such as astaxanthin, absorbability measurement, and stability test were performed based on the descriptions mentioned above. Furthermore, regarding carotenoid-containing compositions 2 and 4 to 6 obtained in Examples 2 and 4 to 6 and the dried crystal obtained in Preparation Example 1, water solubility or water dispersibility were evaluated based on the descriptions mentioned above.

The results are shown in Table 1. Here, as the serum carotenoid concentration, the mean value (n=5) of values 4 hours after administration is shown. As the relative standard deviation (RSD), which shows dispersion, only the serum astaxanthin concentration 4 hours after administration is shown. Smaller RSD means a smaller individual difference in rats (n=5). Particularly, since a carotenoid is known to have a large individual difference in absorbability, it is considered that the carotenoid is absorbed well by everyone (e.g., even by smokers, or persons who genetically poorly absorb a carotenoid) when RSD is small.

TABLE 1

| | | | Preparation | Carotenoid-containing composition | | | | | | |
|---|---|---|---|---|---|---|---|---|---|---|
| | | | Example 1 | 1 | 2 | 3 | 4 | 5 | 6 | 7 |
| Absorbability | 4 hours after | Serum | 3.5 | 10.1 | 153 | 23.8 | 182 | 174 | 192 | 30.5 |
| | administration | astaxanthin | | | | | | | | |
| | | concentration | | | | | | | | |
| | | (ng/mL) | | | | | | | | |
| | | RSD of serum | 33 | 25 | 18 | 21 | 12 | 14 | 13 | 19 |
| | | astaxanthin | | | | | | | | |
| | | concentration | | | | | | | | |
| | | (%) | | | | | | | | |
| | | Serum adonirubin | 0.8 | 1.9 | 29 | 4.6 | 35 | 33 | 37 | 5 |
| | | concentration | | | | | | | | |
| | | (ng/mL) | | | | | | | | |
| | | Serum | 0.5 | 1.3 | 19 | 2.9 | 22 | 21 | 24 | 3.3 |
| | | adonixanthin | | | | | | | | |
| | | concentration | | | | | | | | |
| | | (ng/mL) | | | | | | | | |

| | Fast-acting property | X | Δ | ⊚ | Δ | ⊚ | ⊚ | ⊚ | Δ |
|---|---|---|---|---|---|---|---|---|---|
| | Persistence | X | Δ | Δ | ⊚ | Δ | Δ | Δ | ⊚ |
| Stability | | ⊚ | ⊚ | ◯ | ⊚ | ◯ | ◯ | Δ | ⊚ |
| Water | State | X | — | Δ | — | Δ | Δ | ◯ | — |
| solubility or | Supernatant astaxanthin | <0.1 | — | 3.5 | — | 4.2 | 4 | 5.2 | — |
| Water | concentration (μg/ml) | | | | | | | | |
| dispersibility | | | | | | | | | |

As shown in Table 1 above, carotenoid-containing compositions 1 to 7 had improved absorbability (including fast-acting property and persistence) and smaller RSD, compared with the dried crystal obtained in Preparation Example 1. Particularly, since RSD was reduced, it is considered that the carotenoid-containing composition of the present invention is universally absorbed (i.e., absorbed well by everyone).

Carotenoid-containing compositions 2 and 4 to 6 particularly had effectively improved absorbability and fast-acting property and smaller RSD. Carotenoid-containing compositions 3 and 7 particularly had effectively improved persistence. Carotenoid-containing composition 6 further had improved water solubility, and carotenoid-containing compositions 2, 4, and 5 further had improved water dispersibility.

1. A carotenoid-containing composition obtained by dissolving or dispersing a solid carotenoid derived from a genus Paracoccus microorganism.

2. The composition according to claim 1, wherein the carotenoid comprises one or more carotenoids selected from astaxanthin, adonirubin, adonixanthin, and a pharmaceutically acceptable salt thereof.

3. The composition according to claim 1, wherein the genus Paracoccus microorganism is Paracoccus carotinifaciens.

4. The composition according claim 1, further comprising an orally acceptable or pharmaceutically acceptable additive.

5. The composition according to claim 1 for a human.

6. The composition according to claim 1, which is a food and drink or a food additive or a material thereof.

7. The composition according to claim 1, which is a functional food or a material thereof.

8. The composition according to claim 1, which is a pharmaceutical or a material thereof.

9. A method for improving an absorbability of a carotenoid, which comprises dissolving or dispersing a solid carotenoid derived from a genus Paracoccus microorganism in the presence of an orally acceptable or pharmaceutically acceptable excipient.
